(19) United States
(12) Patent Application Publication
(10) Pub. No.: US 2025/0049841 A1
(43) Pub. Date: Feb. 13, 2025

(54) NITRIC OXIDE-RELEASING NASAL COMPOSITIONS AND METHODS OF USE THEREOF

(71) Applicant: LNHC, INC., Durham, NC (US)

(72) Inventors: Daniel Albert RICCIO, Apex, NC (US); Carri Brodnax GEER, Chapel Hill, NC (US); Benjamin Joseph Monroe PRIVETT, Snow Camp, NC (US); Shaylyn Elizabeth WALTER, Raleigh, NC (US)

(21) Appl. No.: 18/719,012

(22) PCT Filed: Dec. 16, 2022
(86) PCT No.: PCT/US2022/081754

§ 371 Date: Jun. 12, 2024

Related U.S. Application Data
(60) Provisional application No. 63/290,928, filed on Dec. 17, 2021.

(51) Int. Cl. A61K 33/00; A61K 9/00; A61K 47/12; A61P 31/16

(52) U.S. Cl. CPC A61K 33/00; A61K 9/0043; A61K 47/12; A61P 31/16

(57) ABSTRACT

The present invention relates generally to nitric oxide (NO) releasing nasal compositions and to methods of using the same.

### FIELD

The present invention relates generally to nitric oxide (NO)-releasing nasal compositions and to methods of using the same.

### BACKGROUND

Highly pathogenic coronaviruses have seen increased rates of occurrence in the past two decades. The 2003 outbreak of the severe acute respiratory syndrome coronavirus (SARS-CoV) was followed by the Middle East respiratory syndrome coronavirus (MERS-CoV) in 2012. The ongoing epidemic due to severe acute respiratory syndrome coronavirus two (SARS-CoV-2) magnifies the devasting toll that a highly pathogenic and infectious coronavirus can have on a global scale.

Classified as a beta-coronavirus, SARS-CoV-2 is an enveloped, single, positive-stranded RNA virus within the Coronaviridae family. Coronaviruses commonly encode 4 structural proteins, including envelope (E) protein, membrane (M) protein, nucleocapsid (N) protein, and spike (S) protein. While the E and M proteins participate in viral assembly and the N protein encapsulates viral RNA within the virion particle, the S protein is critical in receptor-binding and allows the virion particle to gain entry into host cells and propagate infection. The human angiotensin-converting enzyme 2 (ACE2) receptor has been identified as the predominant host cell access point for both SARS-CoV and SARS-CoV-2. The nasal epithelium is postulated as the major target for viral entry for SARS-CoV-2. Specifically, expression of ACE2 throughout the respiratory tract has been shown to follow a gradient that is greatest in nasal tissue and decreases down the respiratory tract. Due to the high expression of ACE2, the nasal cavity is hypothesized to serve as an initial site for SARS-CoV-2 infection. Indeed, in vitro infection with SARS-CoV-2 is rampant in nasal epithelial cells. Clinically, viral load is highest in nasal and oropharyngeal samples from the upper respiratory tract for those infected with SARS-CoV-2, both asymptomatic and symptomatic. Beyond the respiratory tract, ACE2 expression in numerous tissues reflects the wide array of reported symptoms (e.g., gastrointestinal, vascular) associated with COVID-19. The upper respiratory tract being a large reservoir of viral burden creates a high potential for transmission between individuals and for aspiration of virus-containing droplets to the lower respiratory tract within an individual, which can lead to damage in the lungs (e.g., acute respiratory distress syndrome (ARDS)).

An increased level of rapid infectivity and associated mortality and morbidity has coincided with each outbreak. The increasing frequency of outbreaks of such highly pathogenic coronaviruses underscores the need for treatments such as antiviral treatments to treat current and future emerging pathogens.

### SUMMARY

One aspect of the present invention is directed to a composition comprising: a nitric oxide-releasing active pharmaceutical ingredient; and a buffer configured to maintain the pH of the composition in a range of about 3, 3.5, 4, or 4.5 to about 5, 5.5, 6, 6.5, 7, 7.5, 8, or 8.5.

Another aspect of the present invention is directed to a composition comprising: a nitric oxide-releasing active pharmaceutical ingredient; and a buffer, the buffer comprising a citrate (e.g., a citrate salt) and/or citric acid in an amount of at least 100 mM.

An additional aspect of the present invention is directed to a kit comprising: a first composition comprising a nitric oxide-releasing active pharmaceutical ingredient; and a second composition comprising a buffer configured to maintain the pH of a composition comprising the first and second compositions in a range of about 3, 3.5, 4, or 4.5 to about 5, 5.5, 6, 6.5, 7, 7.5, 8, 8.5, wherein the first composition and second composition are separately stored in the kit.

A further aspect of the present invention is directed to a kit comprising: a first composition comprising a nitric oxide-releasing active pharmaceutical ingredient; and a second composition comprising a buffer comprising a citrate (e.g., a citrate salt) and/or citric acid in an amount of at least 100 mM, wherein the first composition and second composition are separately stored in the kit.

Another aspect of the present invention is directed to a method of treating and/or preventing an infection in a subject, the method comprising administering a composition of the present invention to the subject. In some embodiments, the infection is a viral infection and/or an infection caused by SARS-CoV-2.

It is noted that aspects described with respect to one embodiment may be incorporated in different embodiments although not specifically described relative thereto.

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

### BRIEF DESCRIPTION OF THE DRAWINGS

FIG. 1 is a graph showing the percent initial body weight of 5-week-old golden Syrian hamsters following challenge with SARS-CoV-2 and treatment with berdazimer sodium (n=10 for hamsters group and n=5 for normal controls). Treatment with berdazimer sodium did not prevent weight loss following infection. Treatment with EIDD-2801 at a dose of 200 mg/kg/d prevented weight loss after infection (**P<0.01 compared to placebo-treated hamster).

FIG. 2 is a graph showing lung virus titers of 5-week-old golden Syrian hamsters after challenge with SARS-CoV-2 and treatment with berdazimer sodium. Treatment with berdazimer sodium did not significantly reduce lung virus titers in animals infected with SARS-CoV-2. Treatment with EIDD-2801 reduced lung virus titers on day 6 post-infection (****P<0.0001 compared to placebo-treated hamsters).

FIG. 3 is a graph showing nasal tissue virus titers of 5-week-old golden Syrian hamsters after challenge with SARS-CoV-2 and treatment with berdazimer sodium. Treatment with berdazimer sodium did not significantly reduce nasal tissue virus titers in animals infected with SARS-CoV-2. Treatment with EIDD-2801 did not significantly reduce nasal virus titers after infection.

FIG. 4 is a graph showing oropharyngeal swab virus titers of 5-week-old golden Syrian hamsters after challenge with SARS-CoV-2 and treatment with berdazimer sodium. No significant differences in oropharyngeal swab virus titers were determined by one-way ANOVA.

FIG. 5 is a graph showing lung weights of 5-week-old golden Syrian hamsters after challenge with SARS-CoV-2 and treatment with berdazimer sodium. Lung weights were not statistically different between treatment groups when compared by one-way ANOVA.

FIG. 6 is a graph showing percent initial body weight of 5-week-old golden Syrian hamsters after treatment with berdazimer sodium and cohabitation with SARS-CoV-2-infected hamsters (n=2 hamsters per donor group and n=4 hamsters per naïve group). Placebo-treated naïve animals were protected from weight loss when the donor animals were treated with 2 mg/mL of berdazimer sodium. Treatment with 2 mg/mL of berdazimer sodium prevented weight loss following exposure to SARS-CoV-2-infected hamsters when the donor animals were also treated with 2 mg/mL of berdazimer sodium (*P<0.05, ***P<0.001 compared to berdazimer sodium-treated donor animals).

FIG. 7 is a graph showing day 4 lung virus titers of hamsters treated with berdazimer sodium and exposed to SARS-CoV-2-infected hamsters. Cohabitation of animals occurred on study days 1-3. Treatment with berdazimer sodium significantly reduced lung virus titers naïve animals that were cohabitated with infected animals that were also treated with berdazimer sodium (****P<0.0001 compared to placebo-treated naïve animals).

FIG. 8 is a graph showing day 4 nasal tissue virus titers of hamsters treated with berdazimer sodium and exposed to SARS-CoV-2-infected hamsters. Cohabitation of animals occurred on study days 1-3. Treatment with berdazimer sodium did not significantly reduce nasal tissue virus titers in animals cohabitated with SARS-CoV-2-infected hamsters.

FIG. 9 is a graph showing oropharyngeal swab virus titers of 5-week-old golden Syrian hamsters after treatment with berdazimer sodium and cohabitation with SARS-CoV-2-infected hamsters. Treatment with berdazimer sodium at a dose of 2 mg/mL did not significantly reduce oropharyngeal swab titers in hamsters cohabitated with SARS-CoV-2-infected hamsters.

FIG. 10 is a graph showing lung weights of 5-week-old golden Syrian hamsters after treatment with berdazimer sodium and cohabitation with SARS-CoV-2-infected animals. Lung weights were not statistically different between groups when compared by one-way ANOVA.

FIG. 11 is a graph showing percent initial body weight of 16-week-old golden Syrian hamsters after treatment with berdazimer sodium (8 mg/ml) and cohabitation with SARS-CoV-2-infected hamsters (n=2 hamsters per donor group and n=4 hamsters per naïve group). No statistically significant differences in weight loss were observed following treatment of hamsters with berdazimer sodium (8 mg/ml) and cohabitation with SARS-CoV-2-infected hamsters.

FIG. 12 is a graph showing percent initial body weight of 16-week-old golden Syrian hamsters after treatment with berdazimer sodium (2 or 1 mg/ml) and cohabitation with SARS-CoV-2-infected hamsters (n=2 hamsters per donor group and n=4 hamsters per naïve group). No statistically significant differences in weight loss were observed following treatment of hamsters with berdazimer sodium (2 or 1 mg/ml) and cohabitation with SARS-CoV-2-infected hamsters.

FIG. 13 is a graph showing day 4 lung virus titers of hamsters treated with berdazimer sodium (8 mg/ml) and exposed to SARS-CoV-2-infected hamsters. Cohabitation of animals occurred on study days 1-3. Treatment with berdazimer sodium (8 mg/ml) significantly reduced lung virus titers in naïve animals that were cohabitated with infected animals that were also treated with berdazimer sodium (8 mg/ml) (****P<0.0001 compared to placebo-treated naïve animals).

FIG. 14 is a graph showing day 4 lung virus titers of hamsters treated with berdazimer sodium (2 or 1 mg/ml) and exposed to SARS-CoV-2-infected hamsters. Cohabitation of animals occurred on study days 1-3. Treatment with berdazimer sodium (2 mg/ml) significantly reduced lung virus titers of naïve animals that were cohabitated with infected animals that were also treated with berdazimer sodium (2 mg/ml). Treatment with berdazimer sodium (1 mg/ml) did not significantly reduce lung virus titers in naïve animals that were cohabitated with infected animals that were also treated with berdazimer sodium (1 mg/ml) (****P<0.0001 compared to placebo-treated naïve animals).

FIG. 15 is a graph showing day 4 nasal tissue virus titers of hamsters treated with berdazimer sodium (8 mg/ml) and exposed to SARS-CoV-2-infected hamsters. Cohabitation of animals occurred on study days 1-3. Treatment with berdazimer sodium (8 mg/ml) did not significantly reduce nasal tissue virus titers in animals cohabitated with SARS-CoV-2-infected hamsters.

FIG. 16 is a graph showing day 4 nasal tissue virus titers of hamsters treated with berdazimer sodium (2 or 1 mg/ml) and exposed to SARS-CoV-2-infected hamsters. Cohabitation of animals occurred on study days 1-3. Treatment with berdazimer sodium (2 or 1 mg/ml) did not significantly reduce nasal tissue virus titers in animals cohabitated with SARS-CoV-2-infected hamsters.

FIG. 17 is a graph showing lung weights of 16-week-old golden Syrian hamsters after treatment with berdazimer sodium (8 mg/ml) and cohabitation with SARS-CoV-2-infected animals. Lung weights were not statistically different between groups when compared by one-way ANOVA.

FIG. 18 is a graph showing lung weights of 16-week-old golden Syrian hamsters after treatment with berdazimer sodium (2 or 1 mg/ml) and cohabitation with SARS-CoV-2-infected animals. Lung weights were not statistically different between groups when compared by one-way ANOVA.

FIG. 19 is a graph showing percent initial body weight of hamsters treated once daily with berdazimer sodium (2, 4, or 8 mg/ml) and infected with SARS-CoV-2. Treatment with berdazimer sodium began 24 hours prior to infection. No statistically significant protection from weight loss was observed in hamsters treated with 2, 4, or 8 mg/mL of berdazimer sodium once daily. Although not statistically significant, hamsters treated with 8 mg/mL of berdazimer sodium lost less body weight compared to placebo-treated animals. A similar trend was observed in the hamsters treated with EIDD-2801 at a dose of 500 mg/kg/d.

FIG. 20 is a graph showing lung virus titers at days 3 and 6 post-infection from hamsters treated once daily with berdazimer sodium (2, 4, or 8 mg/ml) and infected with SARS-CoV-2. Treatment with berdazimer sodium began 24 hours prior to infection. Treatment with berdazimer sodium at a dose of 4 mg/mL significantly reduced lung virus titers on day 3 post-infection. Treatment with EIDD-2801 at a dose of 500 mg/kg/d significantly reduced lung virus titers on day 3 post-infection (*P<0.05, **P<0.01 compared to placebo-treated animals).

FIG. 21 is a graph showing nasal tissue virus titers at days 3 and 6 post-infection from hamsters treated with once daily berdazimer sodium (2, 4, or 8 mg/ml) and infected with SARS-CoV-2. Treatment with berdazimer sodium began 24 hours prior to infection. Once daily treatment with berdazimer sodium at doses of 2, 4, or 8 mg/mL did not significantly reduce nasal tissue virus titers. Treatment with EIDD-2801 at a dose of 500 mg/kg/d did not significantly reduce nasal tissue virus titers.

FIG. 22 is a graph showing lung weights at days 3 and 6 post-infection from hamsters treated once daily with berdazimer sodium (2, 4, or 8 mg/ml) and infected with SARS-CoV-2. Treatment with berdazimer sodium began 24 hours prior to infection. Once daily treatment with berdazimer sodium at doses of 2, 4, or 8 mg/mL did not significantly reduce lung weights. Treatment with EIDD-2801 at a dose of 500 mg/kg/d did not significantly reduce lung weights.

FIG. 23 is a graph showing percent initial body weight of hamsters treated twice daily with berdazimer sodium (2 mg/ml) and infected with SARS-CoV-2. Treatment with berdazimer sodium began 24 hours prior to infection. Twice daily treatment with berdazimer sodium at a dose of 2 mg/mL did not prevent weight loss in hamsters infected with SARS-CoV-2. Although not statistically significant, hamsters treated with EIDD-2801 at a dose of 500 mg/kg/d lost less body weight than placebo-treated hamsters.

FIG. 24 is a graph showing lung virus titers at days 3 and 6 post-infection from hamsters treated twice daily with berdazimer sodium (2 mg/ml) and infected with SARS-CoV-2. Treatment with berdazimer sodium began 24 hours prior to infection. Twice daily treatment with berdazimer sodium at a dose of 2 mg/mL did not significantly reduce lung virus titers on day 3 or 6 post-infection. Treatment with EIDD-2801 at a dose of 500 mg/kg/d significantly reduced lung virus titers on day 3 post-infection (**P<0.01 compared to placebo-treated animals).

FIG. 25 is a graph showing nasal tissue virus titers at days 3 and 6 post-infection from hamsters treated twice daily with berdazimer sodium (2 mg/ml) and infected with SARS-CoV-2. Treatment with berdazimer sodium began 24 hours prior to infection. Twice daily treatment with berdazimer sodium at a dose of 2 mg/mL significantly reduce lung virus titers on day 3 post-infection. Treatment with EIDD-2801 at a dose of 500 mg/kg/d significantly reduced nasal tissue virus titers on day 3 post-infection (**P<0.01 compared to placebo-treated animals).

FIG. 26 is a graph showing lung weights at days 3 and 6 post-infection from hamsters treated twice daily with berdazimer sodium (2 mg/ml) and infected with SARS-CoV-2. Treatment with berdazimer sodium began 24 hours prior to infection. Lung weights were significantly affected by twice daily treatment with berdazimer sodium and EIDD-2801 at Day 6 (**P<0.01, ***P<0.0001 compared to placebo-treated animals).

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of±10%, +5%, +1%, +0.5%, or even+0.1% of the specified value as well as the specified value. For example, “about X” where X is the measurable value, is meant to include X as well as variations of±10%, +5%, +1%, +0.5%, or even+0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancing,” “improve” and “improving” (and grammatical variations thereof) describe an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% such as compared to another measurable property or quantity (e.g., a control value). In some embodiments, the reduction can result in no or essentially no (i.e., an insignificant amount, e.g., less than about 10% or even 5%) detectable activity or amount.

According to embodiments of the present invention provided herein are nitric oxide (NO)-releasing compositions. An NO-releasing composition of the present invention may be a nasal composition and/or may be configured for intranasal delivery. In some embodiments, a composition of the present invention comprises a nitric oxide-releasing active pharmaceutical ingredient; and a buffer configured to maintain the pH of the composition in a range of about 3, 3.5, 4, or 4.5 to about 5, 5.5, 6, 6.5, 7, 7.5, 8, 8.5. A buffer can include a weak acid and a salt of the weak acid or a weak base and a salt of the weak base. In some embodiments, a buffer of the present invention comprises a weak acid that has at least two pKa values in a range of about 2.5 to about 6.5. In some embodiments, the weak acid has a pKa value of about 2.5, 3, 3.5, 4, 4.5, 5, 5.5, 6, and/or 6.5. The buffer may have a pH of about 3, 3.5, or 4 to about 4.5, 5, 5.5, or 6 such as about 3, 3.5, 4, 4.5, 5, 5.5, or 6.

Exemplary buffers include, but are not limited to, citrate, acetate, phosphate, and/or maleate buffers. An acetate buffer may comprise acetic acid and/or an acetate (e.g., sodium acetate). A phosphate buffer may comprise phosphoric acid and/or a phosphate (e.g., monobasic dihydrogen phosphate and dibasic monohydrogen phosphate). A maleate buffer may comprise maleic acid and/or a maleate (e.g., tromethamine maleate salt). A citrate buffer may comprise citric acid and/or a citrate (e.g., a citrate salt such as sodium citrate tribasic and/or tri sodium citrate dihydrate). In some embodiments, a composition of the present invention comprises a citrate buffer. A citrate buffer may comprise a citrate ion (e.g., tricarboxylic acid trianion) and/or mono-hydrogen citrate ion.

A buffer of the present invention may have a concentration of an acid and/or a corresponding salt of the acid in an amount that is at least 50 mM or at least 100 mM or more. In some embodiments, a buffer has a concentration of an acid and/or a corresponding salt of the acid in an amount of about 50, 75, 100, 150, 200, 250, 300, 350, 400, 450, 500, 550, 600, 650, 700, 750, 800, 850, 900, 950, or 1000 mM or more. In some embodiments, a buffer has a concentration of an acid and/or a corresponding salt of the acid in an amount of about 50 mM to about 150 mM, about 100 mM to about 300 mM, about 150 mM to about 300 mM, about 200 mM to about 1000 mM, about 200 mM to about 500 mM, about 250 mM to about 600 mM, or about 500 mM to about 1000 mM. In some embodiments, the buffer comprises a citrate (e.g., a citrate salt) and/or citric acid in an amount of about 200 mM or more. In some embodiments, the buffer has a concentration of an acid and/or a corresponding salt of the acid in an amount that would not be expected to be tolerated by a subject and/or suitable for intranasal administration. In some embodiments, the buffer has a concentration of an acid and/or a corresponding salt of the acid in an amount that exceeds what is typically established as tolerable by a subject and/or suitable for intranasal administration.

A buffer of the present invention may comprise at least one additional acid (e.g., HCl) and/or base (e.g., NaOH) in an amount sufficient to adjust the pH of the buffer such as to a pH of about 3, 3.5, or 4 to about 4.5, 5, 5.5, or 6. In some embodiments, the buffer has a pH of about 3, 3.5, 4, 4.5, 5, 5.5, or 6.

In some embodiments, the buffer is a citrate buffer. The citrate buffer may comprise a citrate (e.g., a citrate salt) and/or citric acid. Exemplary citrates include, but are not limited to, trisodium citrate, potassium citrate, calcium citrate, and/or a hydrate thereof and/or anhydrous form thereof. In some embodiments, a citrate buffer comprises a citrate and citric acid. In some embodiments, the citrate is sodium citrate tribasic. In some embodiments, the citrate is tri sodium citrate dihydrate. In some embodiments, a citrate buffer of the present invention comprises citric acid in an amount of about 1%, 1.25%, 1.5%, or 1.75% to about 2%, 20.25%, or 2.5% w/w; sodium citrate tribasic, tri sodium citrate dihydrate, and/or anhydrous sodium citrate in an amount of about 1.5%, 1.75%, 2%, 20.25%, 2.5%, 20.75%, or 3% to about 30.25%, 3.5%, 3.75%, or 4% w/w; optionally an additional acid (e.g., HCl) and/or base (e.g., NaOH) in an amount sufficient to adjust the pH of the buffer to about 3, 3.5, or 4 to about 4.5, 5, 5.5, or 6 (e.g., about 4.5); and a remainder of water. In some embodiments, a citrate buffer of the present invention comprises citric acid in an amount of about 1%, 1.25%, 1.5%, 1.75%, 2%, 2.25%, or 2.5% w/w and sodium citrate tribasic, tri sodium citrate dihydrate, and/or anhydrous sodium citrate in an amount of about 1.5%, 1.75%, 2%, 2.25%, 2.5%, 2.75%, 3%, 3.25%, 3.5%, 3.75%, or 4% w/w, and has a pH of about 4.5.

The nitric oxide-releasing active pharmaceutical ingredient (API) may be suspended in the buffer and/or composition of the present invention. The NO-releasing API may be an insoluble, particulate such as an insoluble, nonbiodegradable particulate. In some embodiments, the NO-releasing API does not comprise a biopolymer and/or is not prepared from a biopolymer. In some embodiments, as nitric oxide is released from a NO-releasing API, the pH of a composition in which the NO-releasing API is present may increase.

In some embodiments, a composition of the present invention comprises a nitric oxide-releasing active pharmaceutical ingredient in an amount of about 0.1 mg/mL to about 30 mg/mL and a buffer configured to maintain the pH of the composition in a range of about 3, 3.5, 4, or 4.5 to about 5, 5.5, 6, 6.5, 7, 7.5, 8, 8.5 (e.g., a citrate buffer). In some embodiments, the buffer is configured to maintain the pH of the composition including the NO-releasing API at about 8.5 or less such as a pH of about 8, 7.5, 7, 6.5, 6, 5.5, 5 or less. In some embodiments, the buffer is configured to maintain the pH of the composition including the NO-releasing API at a pH in a range of about 4.5 to about 5.5 or about 4.5 to about 7. In some embodiments, the NO-releasing API is present in the composition in an amount of about 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mg/mL.

In some embodiments, the NO-releasing API is present in the composition in an amount of about 1 mg/mL to about 20 mg/mL. In some embodiments, the NO-releasing API is present in the composition in an amount of about 2, 4, 6, 8, 10, 12, or 14 mg/mL.

A composition of the present invention may have a pH that varies over time, optionally due to the presence of the NO-releasing API in the composition and/or release of nitric oxide from the NO-releasing API. In some embodiments, the pH of the composition, upon initial formation of the composition (e.g., combining the NO-releasing API and the buffer), is about 4.5 to about 6. The buffer may be configured to maintain the pH of the composition including the NO-releasing API and the buffer in a range of about 3, 3.5, 4, or 4.5 to about 5, 5.5, 6, 6.5, 7, 7.5, 8, or 8.5; in this manner, the composition may have a pH of about 3, 3.5, 4, or 4.5 to about 8.5. In some embodiments, the composition has a pH of about 3, 3.5, 4, 4.5, 5, 5.5, 6, 6.5, 7, 7.5, 8, or 8.5. In some embodiments, the composition has a pH of about 7 or less. In some embodiments, the composition has a pH of about 5.5.

“Nitric oxide-releasing active pharmaceutical ingredient,” “nitric oxide-releasing API,” and “NO-releasing API,” as used herein, refer to a compound or other composition that provides nitric oxide to the skin (e.g., mucosa) and/or tissue of a subject, but is not gaseous nitric oxide. In some embodiments, the NO-releasing API is also not acidified nitrite. In some embodiments, the NO-releasing API includes a nitric oxide-releasing compound, hereinafter referred to as a “NO-releasing compound.” A NO-releasing compound includes at least one NO donor, which is a functional group that may release nitric oxide under certain conditions.

In some embodiments, the NO-releasing compound includes a small molecule compound that includes an NO donor group. “Small molecule compound” as used herein is defined as a compound having a molecular weight of less than 500 Daltons, and includes organic and/or inorganic small molecule compounds. In some embodiments, the NO-releasing compound includes a macromolecule that includes an NO donor group. A “macromolecule” is defined herein as any compound that has a molecular weight of 500 Daltons or greater. In some embodiments, a NO-releasing macromolecule comprises a crosslinked or non-crosslinked polymer, dendrimer, metallic compound, organometallic compound, inorganic-based compound, and/or other macromolecular scaffold. In some embodiments, the macromolecule has a nominal diameter ranging from about 0.1 nm to about 100 μm and may comprise the aggregation of two or more macromolecules, whereby the macromolecular structure is further modified with an NO donor group.

In some embodiments, the NO-releasing compound includes a diazeniumdiolate functional group as an NO donor. The diazeniumdiolate functional group may produce nitric oxide under certain conditions, such as upon exposure to water or a proton. As another example, in some embodiments, the NO-releasing compound includes a nitrosothiol functional group as the NO donor. The NO donor may produce nitric oxide under certain conditions, such as upon exposure to light. Examples of other NO donor groups include nitrosamine, hydroxyl nitrosamine, hydroxyl amine and hydroxyurea. In some embodiments, a combination of NO donors and/or NO-releasing compounds may be present in a composition of the present invention. Additionally, the NO donor may be incorporated into and/or onto the small molecule or macromolecule through covalent and/or non-covalent interactions.

An NO-releasing macromolecule may be in the form of an NO-releasing particle, such as those described in U.S. Pat. Nos. 8,282,967; 8,962,029 or U.S. Pat. No. 8,956,658, the disclosures of which are incorporated by reference herein in their entirety. Other non-limiting examples of NO-releasing compounds include NO-releasing zeolites as described in United States Patent Publication Nos. 2006/0269620 or 2010/0331968; NO-releasing metal organic frameworks (MOFs) as described in United States Patent Application Publication Nos. 2010/0239512 or 2011/0052650; NO-releasing multi-donor compounds as described in International Application No. PCT/US2012/052350 entitled “Tunable Nitric Oxide-Releasing Macromolecules Having Multiple Nitric Oxide Donor Structures”; NO-releasing dendrimers or metal structures as described in U.S. Publication No. 2009/0214618; nitric oxide releasing coatings as described in U.S. Publication No. 2011/0086234; and compounds as described in U.S. Publication No. 2010/0098733. The disclosures of each of the references in this paragraph are incorporated herein by reference in their entirety. Additionally, NO-releasing macromolecules may be fabricated as described in International Application No. PCT/US2012/022048 entitled “Temperature Controlled Sol-Gel Co-Condensation” filed Jan. 20, 2012, the disclosure of which is incorporated herein by reference in its entirety.

As an example, in some embodiments of the present invention, a nitric oxide-releasing active pharmaceutical ingredient may include NO-loaded precipitated silica. The NO-loaded precipitated silica may be formed from nitric oxide donor modified silane monomers into a co-condensed siloxane network. In one embodiment of the present invention, the nitric oxide donor may be an N-diazeniumdiolate. In some embodiments of the present invention, the nitric oxide-releasing active pharmaceutical ingredient may comprise, consist essentially of, or consist of a co-condensed siloxane network comprising a diazeniumdiolate (e.g., a N-diazeniumdiolate).

In some embodiments, the nitric oxide donor may be formed from an aminoalkoxysilane by a pre-charging method, and the co-condensed siloxane network may be synthesized from the condensation of a silane mixture that includes an alkoxysilane and the aminoalkoxysilane to form a nitric oxide donor modified co-condensed siloxane network. As used herein, the “pre-charging method” means that aminoalkoxysilane is “pretreated” or “precharged” with nitric oxide prior to the co-condensation with alkoxysilane. In some embodiments, precharging with nitric oxide may be accomplished by chemical methods. In another embodiment, the “pre-charging” method may be used to create co-condensed siloxane networks and materials more densely functionalized with NO-donors. In some embodiments of the present invention, the nitric oxide-releasing active pharmaceutical ingredient may comprise, consist essentially of, or consist of a co-condensed silica network synthesized from the condensation of a silane mixture comprising an alkoxysilane and at least one aminoalkoxysilane having an amine substituted by a diazeniumdiolate (e.g., a N-diazeniumdiolate).

The co-condensed siloxane network may be silica particles with a uniform size, a collection of silica particles with a variety of size, amorphous silica, a fumed silica, a nanocrystalline silica, ceramic silica, colloidal silica, a silica coating, a silica film, organically modified silica, mesoporous silica, silica gel, bioactive glass, and/or any suitable form or state of silica.

In some embodiments, the alkoxysilane is a tetraalkoxysilane having the formula Si(OR)4, wherein R is an alkyl group. The R groups may be the same or different. In some embodiments the tetraalkoxysilane is selected as tetramethyl orthosilicate (TMOS) or tetraethyl orthosilicate (TEOS). In some embodiments, the aminoalkoxysilane has the formula: R″—(NH—R′)n—Si(OR)3, wherein R is alkyl, R′ is alkylene, branched alkylene, or aralkylene, n is 1 or 2, and R″ is selected from the group consisting of alkyl, cycloalkyl, aryl, and alkylamine.

In some embodiments, the aminoalkoxysilane may be selected from N-(6-aminohexyl)aminopropyltrimethoxysilane (AHAP3); N-(2-aminoethyl)-3-aminopropyltrimethoxysilane (AEAP3); (3-trimethoxysilylpropyl)di-ethylenetriamine (DET3); (aminoethylaminomethyl)phenethyltrimethoxysilane (AEMP3); [3-(methylamino)propyl]trimethoxysilane (MAP3); N-butylamino-propyltrimethoxysilane(n-BAP3); t-butylamino-propyltrimethoxysilane(t-BAP3);N-ethylaminoisobutyltrimethoxysilane(EAiB3); N-phenylamino-propyltrimethoxysilane (PAP3); and N-cyclohexylaminopropyltrimethoxysilane (cHAP3).

In some embodiments, the aminoalkoxysilane has the formula: NH [R′—Si(OR)3]2, wherein R is alkyl and R′ is alkylene. In some embodiments, the aminoalkoxysilane may be selected from bis(3-triethoxysilylpropyl)amine, bis-[3-(trimethoxysilyl)propyl]amine and bis-[(3-trimethoxysilyl)propyl]ethylenediamine.

In some embodiments, as described herein, the aminoalkoxysilane is precharged for NO-release and the amino group is substituted by a diazeniumdiolate. Therefore, in some embodiments, the aminoalkoxysilane has the formula: R″—N(NONO-X+)-R′—Si(OR)3, wherein R is alkyl, R′ is alkylene or aralkylene, R″ is alkyl or alkylamine, and X+ is a cation selected from the group consisting of Na+, K+ and Li+.

The composition of the siloxane network, (e.g., amount or the chemical composition of the aminoalkoxysilane) and the nitric oxide charging conditions (e.g., the solvent and base) may be varied to optimize the amount and duration of nitric oxide release. Thus, in some embodiments, the composition of the silica particles may be modified to regulate the half-life of NO release from silica particles.

In some embodiments, the amino group of aminoalkoxysilane is substituted with a diazeniumdiolate, and the aminoalkoxysilane having a formula of R″—N(NONO-X+)-R′—Si(OR)3, wherein: R is alkyl, R′ is alkylene or aralkylene, R″ is alkyl or alkylamine, and X+ is a cation selected from the group consisting of Na+ and K+.

In some embodiments, the NO-releasing API may comprise a co-condensed silica network comprising and/or formed from diazeniumdiolated aminoethylaminopropyl trimethoxy silane (AEAP3-NONOate) and tetra methyl orthosilicate (TMOS) and/or a co-condensed silica network comprising and/or formed from diazeniumdiolated aminoethylaminopropyl trimethoxy silane (AEAP3-NONOate) and tetraethyl orthosilicate (TEOS). In some embodiments, the NO-releasing API may comprise a co-condensed silica network comprising and/or formed from diazeniumdiolated methylaminopropyl trimethoxysilane (MAP3-NONOate) and tetra methyl orthosilicate (TMOS) and/or a co-condensed silica network comprising and/or formed from diazeniumdiolated methylaminopropyl trimethoxysilane (MAP3-NONOate) and tetraethyl orthosilicate (TEOS). In some embodiments, the NO-releasing API may comprise a co-condensed silica network comprising and/or formed from diazeniumdiolated methylaminopropyl trimethoxysilane (MAP3-NONOate), ethylaminoisobutylsiloxane (EAIB3), and tetraethyl orthosilicate (TEOS). In some embodiments, the NO-releasing API may comprise a co-condensed silica network comprising and/or formed from diazeniumdiolated ethylaminoisobutylsiloxane (EAIB3-NONOate) and tetraethyl orthosilicate (TEOS) and/or tetra methyl orthosilicate (TMOS). In some embodiments, the NO-releasing API may be ethylaminoisobutylsiloxane/methylaminopropylsiloxane-co-polysiloxane (EAIB3:MAP3-NONOate/TEOS). In some embodiments, the NO-releasing API may comprise an amorphous polymer.

In some embodiments, the particle size (e.g., diameter) of a NO-releasing API may be in a range of about 20 nm to about 20 μm or any range therein, such as, but not limited to, about 100 nm to about 20 μm or about 1 μm to about 20 or 30 μm. The particle size may be tailored to minimize or prevent toxicity and/or penetration through the epidermis (or compromised dermis) and into the blood vessels. In some embodiments, the particle size is distributed around a mean particle size (e.g., mean diameter) of less than 20 μm, or any range therein, and the size may allow the particle to enter a follicle. In some embodiments, a NO-releasing API may have a particle size that is distributed around a mean particle size of about 20, 19, 18, 17, 16, 15, 14, 13, 12, 11, 10, 9, 8, 7, 6, 5, 4, 3, 2, or 1 μm. In some embodiments, a NO-releasing API may have a particle size that is distributed around a mean particle size of less than 10 μm, or any range therein, such as, but not limited to about 2 μm to about 10 μm or about 4 μm to about 8 μm. In some embodiments, the particle size may be distributed around a mean particle size of greater than 20 μm, or any range therein, and the size may prevent the particle from entering a follicle. In some embodiments, a mixture of particles with mean particle sizes distributed around two or more mean particle sizes may be provided. A NO-releasing API may be micronized (e.g., ball and/or jet milled). Methods for providing a desired particle size and/or micronization include, but are not limited to, those described in U.S. Patent Application Publication No. 2013/0310533, which is incorporated herein by reference in its entirety. In some embodiments, the nitric oxide-releasing active pharmaceutical ingredient has a mean particle size of about 20 nm to about 30 μm. In some embodiments, the nitric oxide-releasing active pharmaceutical ingredient has a mean particle size of about 2 μm to about 20 μm.

In some embodiments, a NO-releasing API may have a low charge. In some embodiments, charge on a NO-releasing API may be controlled and/or modulated.

A composition of the present invention may comprise a NO-releasing API and may store and/or release nitric oxide in an amount of about 0.001% to about 10% by weight of the composition, such as, but not limited to, about 0.001% to about 0.05%, about 0.01% to about 0.1%, about 0.15% to about 2%, about 0.15% to about 1%, about 0.3% to about 1.2%, about 0.15% to about 6%, about 1% to about 10%, about 3% to about 6%, or about 1% to about 5% by weight of the composition. In some embodiments, a composition of the present invention may comprise a nitric oxide-releasing active pharmaceutical and may store and/or release nitric oxide in an amount of about 0.001%, 0.005%, 0.01%, 0.05%, 0.1%, 0.15%, 0.3%, 0.6%, 0.9%, 1%, 1.25%, 1.5%, 1.75%, 2%, 20.25%, 2.5%, 20.75%, 3%, 30.25%, 3.5%, 3.75%, 4%, 40.25%, 4.5%, 4.75%, 5%, 5.25%, 5.5%, 5.75%, 6%, 6.25%, 6.5%, 6.75%, 7%, 7.25%, 7.5%, 7.75%, 8%, 8.25%, 8.5%, 8.75%, 9%, 9.25%, 9.5%, 9.75%, or 10% by weight of the composition. The amount of nitric oxide released may be determined using real time in vitro release testing. In some embodiments, nitric oxide release may be determined using a chemiluminescent nitric oxide analyzer.

A composition of the present invention may include one or more excipient(s). In some embodiments, the composition comprises a flavoring agent. In some embodiments, the buffer and/or composition is/are devoid of a diluent (e.g., an additional diluent), a co-solvent, a preservative, an antioxidant, a suspending agent, a penetration enhancer, a surfactant, a viscosity-increasing agent, a humectant, a stabilizer, and/or a wetting agent.

Also provided according to embodiments of the present invention are kits. A kit of the present invention may include a first composition comprising a nitric oxide-releasing active pharmaceutical ingredient; and a second composition comprising a buffer of the present invention, wherein the first composition and second composition are separately stored in the kit. In some embodiments, the buffer is configured to maintain the pH of a composition comprising the first composition and second composition (e.g., a combined combination) in a range of about 3, 3.5, 4, or 4.5 to about 5, 5.5, 6, 6.5, 7, 7.5, 8, or 8.5. In some embodiments, the buffer is a citrate buffer such as a citrate buffer comprising a citrate and/or citric acid in an amount of at least 100 mM.

The first composition comprising the nitric oxide-releasing active pharmaceutical ingredient may be a solid and/or the nitric oxide-releasing active pharmaceutical ingredient may be in a particulate form. The second composition may be a solution. In some embodiments, the kit is configured to combine the first composition and the second composition to provide a combined composition. The combined composition can be a composition as described herein. In some embodiments, the kit is configured to provide a combined composition that comprises the nitric oxide-releasing active pharmaceutical ingredient in an amount of about 0.1 mg/mL to about 30 mg/mL and the buffer (e.g., a citrate buffer) is configured to maintain the pH of the combined composition in a range of about 3, 3.5, 4, or 4.5 to about 5, 5.5, 6, 6.5, 7, 7.5, 8, or 8.5.

In some embodiments, the kit is configured to administer and/or release the composition. In some embodiments, a kit of the present invention comprises a device configured for administering the composition. A kit and/or device may administer and/or release a volume of about 15, 25, 50, 75, or 100 μL to about 150, 200, 300, 400, or 500 μL. In some embodiments, the kit and/or device is configured to administer and/or release a volume of about 15, 25, 50, 75, 100, 150, 200, 250, 300, 350, 400, 450, or 500 μL. In some embodiments, the kit and/or device is configured to aerosolize and/or atomize a composition of the present invention (e.g., a composition comprising the first and second compositions). In some embodiments, an aerosolized and/or atomized composition of the present invention has a mean droplet size (e.g., droplet diameter) of about 10, 20, 30, or 40 μm to about 50, 60, 70, 80, 90, or 100 μm. In some embodiments, an aerosolized and/or atomized composition of the present invention has a mean droplet size (e.g., droplet diameter) of about 10, 20, 30, 40, 50, 60, 70, 80, 90, or 100 μm. In some embodiments, an aerosolized and/or atomized composition of the present invention has a mean droplet size that limits or avoids inhalation of the droplets/aerosol into the lungs.

A composition of the present invention may be configured for intranasal administration. In some embodiments, a composition of the present invention is configured to be sprayed and/or is a sprayable composition. The composition may be sprayed onto nasal mucosa and/or oral mucosa. In some embodiments, the composition may contact oropharyngeal tissue. In some embodiments, a composition of the present invention may be configured to be aerosolized and/or atomized and/or may be in the form of an aerosol and/or in the form of small droplets in a gas phase. In some embodiments, a composition of the present invention is configured to be administered to the nasal cavity of a subject and/or to oral mucosa of a subject. The NO-releasing API may be locally delivered to the subject (e.g., to the nasal cavity and/or oral mucosa of the subject). In some embodiments, the local delivery of the NO-releasing API has a local effect and/or a systemic effect.

A composition of the present invention may be antiviral, antimicrobial, and/or antibacterial. A buffer of the present invention may not be antiviral and/or is not viricidal. In some embodiments, a composition of the present invention administers and/or delivers nitric oxide in an amount sufficient to induce apoptosis in cells infected with a pathogen (e.g., a virus and/or bacteria). In some embodiments, a composition of the present invention administers and/or delivers nitric oxide in an amount sufficient to induce apoptosis in virally infected cells. In some embodiments, a composition of the present invention administers and/or delivers nitric oxide in an amount sufficient to reduce or eliminate viral replication, optionally with less than about 50% host cell cytotoxicity.

According to some embodiments, provided is a method of treating and/or preventing an infection (e.g., a viral infection and/or bacterial infection) in a subject, the method comprising administering a composition of the present invention to the subject. In some embodiments, the composition is administered to the subject by applying and/or spraying the composition onto nasal mucosa and/or oral mucosa of a subject. In some embodiments, the administering comprises intranasally administering the composition to the subject.

In some embodiments, the method comprises, prior to administering the composition to the subject, combining a nitric oxide-releasing active pharmaceutical ingredient and a buffer of the present invention to provide the composition. Combining the nitric oxide-releasing active pharmaceutical ingredient and the buffer may comprise adding the NO-releasing API to the buffer or adding the buffer to the NO-releasing API. In some embodiments, the nitric oxide-releasing active pharmaceutical ingredient and buffer are present in a device (e.g., a kit) that is configured to combine the nitric oxide-releasing active pharmaceutical ingredient and the buffer. In some embodiments, the NO-releasing API and buffer are combined and then provided in a device for administration. In some embodiments, a kit of the present invention may be a device configured for administration of the composition. A device of the present invention may be for single use or multiple use. In some embodiments, the device is refillable. In some embodiments, the device is a nasal spray device and/or an atomization device. In some embodiments, the device comprises a syringe and a plug at the tip of the syringe that is configured to atomize the composition as it moves from the syringe and through the plug.

In some embodiments, administering the composition comprises administering a volume of about 15, 25, 50, 75, or 100 μL to about 150, 200, 300, 400, or 500 μL of the composition to the subject. A volume of about 15, 25, 50, 75, or 100 μL to about 150, 200, 300, 400, or 500 μL of the composition may be administered to each nostril of a subject per dose. In some embodiments, the method comprises administering a volume of about 15, 25, 50, 100, 150, 200, 250, 300, 350, 400, 450, or 500 μL of the composition/dose.

A method of the present invention may comprise administering a composition of the present invention to a subject at a time in which the composition has a pH of about 3, 3.5, 4, or 4.5 to about 5, 5.5, 6, 6.5, 7, 7.5, 8, or 8.5. In some embodiments, the composition is administered to the subject at a time in which the composition has a pH of about 5.5.

A method of the present invention may comprise administering and/or delivering exogenous, gaseous nitric oxide to the upper respiratory tract, lower respiratory tract, and/or lungs of a subject. The exogenous, gaseous NO may be delivered to the upper respiratory tract, lower respiratory tract, and/or lungs upon administering a composition of the present invention to the nasal mucosa and/or oral mucosa of the subject. In some embodiments, the NO-releasing API and/or composition (e.g., aerosolized composition) are not delivered to the lower respiratory tract and/or to a lung of the subject. In some embodiments, the NO-releasing API and/or composition are locally delivered to the subject.

A method of the present invention may reduce or prevent transmission of a pathogen to an uninfected subject, optionally compared to the amount of transmission in the absence of a method of the present invention. In some embodiments, the method reduces or prevents transmission of a virus (i.e., viral transmission) to an uninfected subject, optionally compared to the amount of viral transmission in the absence of a method of the present invention.

In some embodiments, a method of the present invention reduces the amount of a pathogen (e.g., a virus, bacteria, etc.) present in a subject (e.g., in a nasal cavity and/or lung of the subject) by at least about 30%, 40%, 50%, 60%, 70%, 80%, 90%, or 100% compared to the initial amount of the virus present in the subject. In some embodiments, a method of the present invention reduces the amount of a pathogen (e.g., a virus, bacteria, etc.) present in the lung(s) of the subject, optionally compared to the amount of the pathogen present in the lung(s) of the subject in the absence of a method of the present invention. In some embodiments, a method of the present invention reduces or prevents progression of pathogen and/or infection (e.g., a viral infection) into the lung(s) of the subject, optionally compared to the progression of the pathogen and/or infection into the lung(s) of the subject in the absence of a method of the present invention. A method of the present invention may reduce the severity of an infection (e.g., a viral infection), optionally compared to the severity in the absence of a method of the present invention.

A composition and/or method of the present invention may reduce or prevent growth and/or replication of a pathogen (e.g., a virus). In some embodiments, the composition and/or method reduces or prevents viral replication. In some embodiments, the composition and/or method reduces or prevents bacterial growth. The method may reduce or inhibit replication of the pathogen (e.g., virus) by disrupting a protein function. In some embodiments, a composition and/or method of the present invention may reduce or prevent shedding of a virus, optionally compared to a method in the absence of the present invention. In some embodiments, a method of the present invention increases oxygenation in the blood of a subject, optionally compared to the oxygen level in the blood prior to administration. In some embodiments, the method may provide a local and/or transient increase in oxygen in the blood of the subject.

In some embodiments, the pathogen is a virus or bacteria. A method of the present invention may treat and/or prevent an infection caused by the pathogen. The infection may be a nosocomial infection. In some embodiments, a method of the present invention may treat and/or prevent an infection that is caused by a pathogen selected from a Coronaviridae virus, Staphylococcus aureus, influenza, and/or respiratory syncytial virus (RSV). In some embodiments, the pathogen is a Coronaviridae virus such as, but not limited to, severe acute respiratory syndrome coronavirus (SARS-CoV), Middle East respiratory syndrome coronavirus (MERS-CoV), severe acute respiratory coronavirus 2 (SARS-CoV-2), a common cold virus (e.g., hCoV-229E and/or hCoV-OC43) and/or a variant thereof. Exemplary variants of SARS-CoV-2 include, but not limited to, SARS-CoV-2 Alpha variant, SARS-CoV-2 Beta variant, SARS-CoV-2 Gamma variant, SARS-CoV-2 Delta variant, SARS-CoV-2 Omicron variant, and/or a variant thereof (e.g., an emerging variant). In some embodiments, a method of the present invention treats and/or prevents infection by a Coronaviridae virus such as SARS-CoV-2 and/or treats and/or prevents a coronavirus disease (e.g., COVID-19). In some embodiments, the pathogen is an influenza virus, such as Influenza A/California/7/2009 (H1N1) and/or a variant thereof. In some embodiments, a method of the present invention treats and/or prevents infection by an influenza virus, such as H1N1, and/or treats and/or prevents an influenza disease. In some embodiments, the pathogen is a respiratory syncytial virus, such as Respiratory syncytial virus strain A2 (RSV-A2) and/or a variant thereof. In some embodiments, a method of the present invention treats and/or prevents infection by a respiratory syncytial virus, such as RSV-A2), and/or treats and/or presents a respiratory syncytial viral disease.

A method of the present invention may comprise administering a composition of the present invention one or more (e.g., 1, 2, 3, 4, 5, 6, 7, or 8) times a day, optionally for 1, 2, 3, 4, 5, 6, 7, 8, 9, 10, 11, 12, 13, 14, or more days. In some embodiments, the composition is administered one, two, or three times a day for about 7 to about 14 days. The method may comprise administering the composition to one or both nostrils of a subject optionally one, two, or three times a day for a period of time (e.g., 1 to 14 or more days).

In some embodiments, a composition and/or method of the present invention provides a localized, topical treatment that can reduce pathogen burden (e.g., viral burden) in the nasal and/or oral epithelium and/or lung(s) of an infected subject and/or may reduce shedding (e.g., viral shedding) and/or transmission of the pathogen to another subject (e.g., an uninfected subject). A composition and/or method of the present invention may treat and/or reduce one or more symptom(s) of an infection and/or may disrupt progression of a disease (e.g., infection) before the disease spreads to the lower respiratory tract of a subject.

A composition and/or method of the present invention may treat and/or prevent an infection (e.g., a SARS-CoV-2 infection) in a subject by disrupting an ACE2 and/or S protein interaction and/or activity. In some embodiments, a composition and/or method of the present invention may inhibit bruton tyrosine kinase (BTK) and/or NF-κB and/or inhibit formation of the NLRP3 inflammasome. In some embodiments, a composition and/or method of the present invention may disrupt a protease function (e.g., a viral protease function) necessary for pathogen replication (e.g., viral replication).

“Treat,” “treating” or “treatment of” (and grammatical variations thereof) as used herein refer to any type of treatment that imparts a benefit to a subject and may mean that the severity of the subject's condition is reduced, at least partially improved or ameliorated and/or that some alleviation, mitigation or decrease in at least one clinical symptom associated with the condition (e.g., a viral infection) is achieved and/or there is a delay in the progression of the condition. In some embodiments, the severity of a condition such as, e.g., a viral infection (e.g., a viral infection caused by SARS-CoV-2), may be reduced in a subject compared to the severity of the condition in the absence of a method of the present invention. In some embodiments, a method of the present invention may treat a viral infection by eliminating at least one clinical symptom associated with the viral infection for a given period of time (e.g., 1, 2, 3, 4, 5, or 6 day(s), or 1, 2, 3, 4, or more weeks, etc.).

In some embodiments, a composition of the present invention is administered in a treatment effective amount. A “treatment effective amount” and “therapeutically effective amount” are used interchangeably herein and refer to an amount that is sufficient to treat (as defined herein) a subject. Those skilled in the art will appreciate that the therapeutic effects need not be complete or curative, as long as some benefit is provided to the subject. In some embodiments, a treatment effective amount of a composition of the present invention may be administered and may include administering a treatment effective amount of a nitric oxide-releasing active pharmaceutical ingredient. In some embodiments, a treatment effective amount of nitric oxide may be administered and/or applied in a method of the present invention. In some embodiments, a method of the present invention is carried out in a manner such that the administration of a composition comprising a nitric oxide (NO)-releasing active pharmaceutical ingredient (API) does not produce systemic effects (e.g., adverse systemic effects) from the administration of nitric oxide, such as, for example, when the composition, NO-releasing API, and/or NO is administered in a treatment effective amount.

The terms “prevent,” “preventing” and “prevention” (and grammatical variations thereof) refer to avoidance, reduction and/or delay of the onset of a condition (e.g., a viral infection) and/or a clinical symptom associated therewith in a subject and/or a reduction in the severity of the onset of the condition and/or clinical symptom relative to what would occur in the absence of a method of the present invention. The prevention can be complete, e.g., the total absence of the condition and/or clinical symptom. The prevention can also be partial, such that the occurrence of the condition and/or clinical symptom in the subject and/or the severity of onset is less than what would occur in the absence of a method of the present invention. In some embodiments, a method of the present invention prevents a viral infection in a subject, such as a viral infection that is caused by SARS-CoV-2.

In some embodiments, a composition of the present invention is administered in a prevention effective amount. A “prevention effective” amount as used herein is an amount that is sufficient to prevent (as defined herein) the condition (e.g., viral infection) and/or clinical symptom in the subject. Those skilled in the art will appreciate that the level of prevention need not be complete, as long as some benefit is provided to the subject. In some embodiments, a prevention effective amount of a composition of the present invention may be administered and may include administering a prevention effective amount of a nitric oxide-releasing active pharmaceutical ingredient. In some embodiments, a prevention effective amount of nitric oxide may be administered and/or applied in a method of the present invention. In some embodiments, a method of the present invention is carried out in a manner such that the administration of a composition comprising a NO-releasing API does not produce systemic effects (e.g., adverse systemic effects) from the administration of nitric oxide, such as, for example, when the composition, NO-releasing API, and/or NO is administered in a prevention effective amount.

The present invention finds use in both veterinary and medical applications. Suitable subjects of the present invention include, but are not limited to, avians and mammals. The term “avian” as used herein includes, but is not limited to, chickens, ducks, geese, quail, turkeys, pheasants, parrots, parakeets, macaws, cockatiels, canaries, and finches. The term “mammal” as used herein includes, but is not limited to, primates (e.g., simians and humans), non-human primates (e.g., monkeys, baboons, chimpanzees, gorillas), bovines, ovines, caprines, ungulates, porcines, equines, felines, canines, lagomorphs, pinnipeds, rodents (e.g., rats, hamsters, and mice), etc. In some embodiments, the subject is a mammal and in certain embodiments the subject is a human. Human subjects include both males and females and subjects of all ages including fetal, neonatal, infant, juvenile, adolescent, adult, and geriatric subjects.

The methods of the present invention may also be carried out on animal subjects, particularly mammalian subjects such as mice, rats, dogs, cats, livestock and horses for veterinary purposes, and/or for drug screening and drug development purposes.

In some embodiments, the subject is “in need of” or “in need thereof” a method of the present invention, for example, the subject is in an at-risk population (e.g., the subject may be at-risk for or more susceptible to a viral infection), the subject has findings typically associated with a viral infection, and/or the subject is suspected to be or to have been exposed to a virus. In some embodiments, a subject in need thereof has a viral infection and/or a clinical sign or symptom associated therewith that may be treated with a method of the present invention. The present invention may be particularly suitable for children, adolescents, adults, and/or geriatric subjects.

In some embodiments, a method of the present invention may administer nitric oxide to the basal layer of a subject's epithelium. A method of the present invention may administer a treatment effective and/or a prevention effective amount of nitric oxide to the basal layer of a subject's epithelium. In some embodiments, nitric oxide may be administered to the basement membrane of a subject's epithelium.

In some embodiments, a method of the present invention may administer nitric oxide in an amount sufficient to induce apoptosis or other cellular damage in virally infected cells.

In some embodiments, a method of the present invention may administer nitric oxide in an amount sufficient to inhibit and/or prevent viral replication in virally infected cells. A method of the present invention may reduce viral replication by at least about 10%, 15%, 20%, 25%, 30%, 35%, 40%, 45%, 50%, 55%, 60%, 65%, 70%, 75%, 80%, 85%, 90%, 95%, 98%, 99%, or 100% compared to the rate of replication prior to the method of the present invention.

In some embodiments, a method of the present invention may treat and/or prevent a viral infection in a subject without cytotoxicity to host cells or with reduced cytotoxicity to host cells. The method may treat and/or prevent the viral infection in the subject with reduced host cell cytotoxicity compared to a different method for treating the viral infection, such as, for example, one that does not administer nitric oxide to the skin and/or tissue of a subject or one that uses acidified nitrite. In some embodiments, a method of the present invention may reduce host cell cytotoxicity by at least about 10%, 15%, 20%, 25%, 30%, 35%, 40%, 45%, 50%, 55%, 60%, 65%, 70%, 75%, 80%, 85%, 90%, 95%, 98%, 99%, or 100% compared to a different method for treating the viral infection. A method of the present invention may reduce and/or eliminate viral replication with no or minimal host cell cytotoxicity. For example, the method may provide a host cell cytotoxicity of about 50% or less (e.g., about 40%, 30%, 20%, 10%, 5%, or less). Cytotoxicity may be determined using methods known to those of skill in the art, such as, for example, a qualitative reading of hematoxylin & eosin (H&E) slides, a lactate dehydrogenase (LDH) assay and/or a 3-(4, 5-Dimethyl-2-thiazolyl)-2, 5-diphenyl-2H-tetrazolium bromide (MTT) assay. In some embodiments, a method of the present invention may not cause apoptosis. For example, the method may not cause apoptosis in keratinocyte layers of the skin and/or tissue.

In some embodiments, a method of the present invention may reduce the amount of viral DNA in virally infected cells of a subject and/or in the nasal cavity of a subject. For example, a method of the present invention may reduce the amount of viral DNA (e.g., in virally infected cells) by at least about 10%, 15%, 20%, 25%, 30%, 35%, 40%, 45%, 50%, 55%, 60%, 65%, 70%, 75%, 80%, 85%, 90%, 95%, 98%, 99%, or 100% compared to the amount of viral DNA present prior to the method of the present invention.

The present invention is explained in greater detail in the following non-limiting Examples.

### EXAMPLES

### Example 1

The antiviral activity of berdazimer sodium (a polysiloxane, polymeric scaffold including tetraethoxysilane (TEOS), N-methylaminopropyl trimethoxysilane (MAP3), and N, N-methylaminopropyl diazeniumdiolate trimethoxysilane (MAP3-NONOate)) and MAP3-NONOate were evaluated against SARS-CoV-2 (strain USA-WA1/2020) in a highly differentiated, three-dimensional (3-D), in vitro model of normal, human-derived tracheal/bronchial epithelial (TBE) cells. The compounds were tested at 4 concentrations in singlet or duplicate inserts of the 3D tissue models of the human airway (MatTek Life Sciences) as indicated in Table 1. Antiviral activity was measured by virus yield reduction assays 5 days after infection.

### Materials and Methods

Compounds: The test compounds were provided as solids and stored at −20° C. upon arrival. A single aliquot of the compound Berdazimer Sodium was dissolved in 100% DMSO at concentrations of 200, 160, 80, and 20 mg/mL just prior to the start of the experiment then further diluted to the test dilutions in the MatTek culture medium (AIR-100-MM). A single aliquot of the compound MAP3-NONOate was dissolved in 100% methanol at concentrations of 200, 160, 80, and 20 mg/mL the morning of the experiment then further diluted to the test dilutions in the MatTek culture medium. Remdesivir (MedChemExpress, cat #HY-104077) was tested in singlet wells at 1, 0.1, 0.01, and 0.001 μg/mL as the positive control.

Cell Culture: The EpiAirway™ Model consists of normal, human-derived tracheal/bronchial epithelial (TBE) cells which have been cultured to form a multi layered, highly differentiated model which closely resembles the epithelial tissue of the respiratory tract.

The cell cultures were made to order by MatTek Life Sciences (https://www.mattek.com) (Ashland, MA) and arrived in kits with either 12- or 24-well inserts each. The TBE cells were grown on 6 mm mesh disks in transwell inserts. During transportation the tissues were stabilized on a sheet of agarose, which was removed upon receipt. One insert was estimated to consist of approximately 1.2×106 cells. Kits of cell inserts (EpiAirway™ AIR-100, AIR-112) originated from a single donor, #9831, a 23-year old, healthy, non-smoking, Caucasian male. The cells have unique properties in forming layers, the apical side of which is exposed only to air and that creates a mucin layer. Upon arrival, the cell transwell inserts were immediately transferred to individual wells of a 6-well plate according to manufacturer's instructions, and 1 mL of MatTek's proprietary culture medium (AIR-100-MM) was added to the basolateral side, whereas the apical side was exposed to a humidified 5% CO2 environment. The TBE cells were cultured at 37° C. for one day before the start of the experiment. After the 24 h equilibration period, the mucin layer, secreted from the apical side of the cells, was removed by washing with 400 μL pre-warmed 30 mM HEPES buffered saline solution 3X. Culture medium was replenished following the wash steps.

Viruses: SARS-CoV-2 strain USA-WA1/2020 was passaged three times in Vero 76 cells to create the virus stock. Virus was diluted in AIR-100-MM medium before infection, yielding a multiplicity of infection (MOI) of approximately 0.01 CCID50 per cell.

Experimental design: Each 2× compound treatment (120 L) and virus (120 μL) was applied to the apical side, and 1× compound treatment only was applied to the basal side (1 mL), for a 2 h incubation. As virus controls, 3 of the cell wells were treated with the Berdazimer sodium medium (cell culture medium containing 0.5% DMSO), 3 wells were treated with the MAP3-NONOate medium (cell culture medium containing 0.3% methanol, and 3 wells were treated with cell culture medium only as the Remdesivir virus control. Following the 2 h infection, the apical medium was removed, and the basal side was replaced with freshly prepared compound or medium. The cells were maintained at the air/liquid interface. On days 1, 2, 3, and 4 post-infection, the test compounds were prepared fresh, drug was removed from the duplicate wells and treated with fresh drug. The basal side of the singlet wells were replaced with fresh growth medium containing the DMSO or methanol only, no test compound. On day 5, the medium was removed and discarded from the basal side. Virus released into the apical compartment of the tissues was harvested by the addition of 400 μL of culture medium that was pre-warmed at 37° C. The contents were incubated for 30 min, mixed well, collected, thoroughly vortexed and plated on Vero 76 cells for VYR titration. Triplicate and singlet wells were used for virus control and cell controls, respectively.

Determination of virus titers from each treated cell culture: Vero 76 cells were seeded in 96-well plates and grown overnight (37° C.) to 90% confluence. Samples containing virus were diluted in 10-fold increments in infection medium and 200 μL of each dilution transferred into respective wells of a 96-well microtiter plate. Four microwells were used for each dilution to determine 50% viral endpoints. After 7 days of incubation, each well was scored positive for virus if any cytopathic effect (CPE) was observed as compared with the uninfected control, and counts were confirmed for endpoint on day 10. The virus dose that was able to infect 50% of the cell cultures (CCID50 per 0.1 mL) was calculated by the Reed-Muench method (Reed, L. J., Muench, H., 1938. A simple method of estimating fifty percent endpoints. The American Journal of Hygiene 27, 493-497). The day 7 values are reported. Untreated, uninfected cells were used as the cell controls.

### Results

The virus yield results and EC90 values are summarized in Table 1. Once daily repeat treatments for each compound (Berdazimer Sodium and MAP3-NONOate) demonstrated an antiviral response while the single treatment regimen for either did not under the conditions tested. No toxicity was observed at any of the test compound concentrations.

TABLE 1
Antiviral efficacy: EC90 for Berdazimer sodium
and MAP3-NONOate against SARS-CoV-2.

| | | | | | | | Titer | |
|---|---|---|---|---|---|---|---|---|
| Drug name | Conc. | | Titer 1 | EC₉₀ | Titer 2 | EC₉₀ | Avg. | EC₉₀ |
| DMSO | MOI 0.01 | | 4.50 | | | | 4.33 | |
| SARS-CoV-2 | | | 4.50 | | | | | |
| virus control | | | 4.00 | | | | | |
| drug replaced daily | 1 | mg/ml | 2.67 | 0.64 | 3.00 | 0.86 | 2.84 | 0.75 |
| Berdazimer Sodium | 0.8 | | 3.00 | | 3.50 | | 3.25 | |
| | 0.4 | | 4.00 | | 3.67 | | 3.84 | |
| | 0.1 | | 3.67 | | 3.50 | | 3.59 | |
| media replaced daily | 1 | mg/ml | 3.30 | 0.99 | | | 3.30 | 0.99 |
| Berdazimer Sodium | 0.8 | | 4.00 | | | | 4.00 | |
| | 0.4 | | 4.30 | | | | 4.30 | |
| | 0.1 | | 4.30 | | | | 4.30 | |
| Methanol | MOI 0.01 | | 4.00 | | | | 4.27 | |
| SARS-CoV-2 | | | 4.30 | | | | | |
| virus control | | | 4.50 | | | | | |
| drug replaced daily | 0.6 | mg/ml | 2.50 | 0.51 | 3.00 | 0.44 | 2.75 | 0.48 |
| MAP3-NONOate | 0.48 | | 3.30 | | 3.30 | | 3.30 | |
| | 0.24 | | 3.67 | | 3.67 | | 3.67 | |
| | 0.06 | | 3.00 | | 4.00 | | 3.50 | |
| media replaced daily | 0.6 | mg/ml | 3.30 | >0.6 | | | 3.30 | >0.6 |
| MAP3-NONOate | 0.48 | | 3.67 | | | | 3.67 | |
| | 0.24 | | 4.30 | | | | 4.30 | |
| | 0.06 | | 4.67 | | | | 4.67 | |
| growth medium | MOI 0.005 | | 4.30 | | | | 4.20 | |
| SARS-CoV-2 | | | 4.30 | | | | | |
| virus control | | | 4.00 | | | | | |
| Remdesivir | 1 | ug/mL | 1.67 | 0.0065 | | | 1.67 | 0.0065 |
| | 0.1 | ug/mL | 2.50 | | | | 2.50 | |
| | 0.01 | ug/mL | 3.30 | | | | 3.30 | |
| | 0.001 | ug/ml | 3.50 | | | | 3.50 | |

Each well was scored positive for virus if any CPE was observed as compared with the uninfected control. Vero 76 cells were scored on day 7 and confirmed on day 10.
^{a}Titer results from the virus yield reduction assay.
^{b}EC90 = 90% effective concentration (concentration to reduce virus yield by 1 log₁₀) determined by regression analysis.

### Example 2

The objective of this study was to assess the efficacy of berdazimer sodium for treatment of a SARS-CoV-2 infection in wild-type golden Syrian hamsters. In addition, the efficacy of berdazimer sodium at reducing the direct transmission of SARS-CoV-2 from infected animals to naïve littermates was also evaluated.

The effect of berdazimer sodium treatment on weight loss, lung virus titers, nasal tissue titers, lung weights, and oropharyngeal swab titers in hamsters exposed to SARS-CoV-2 were the primary endpoints.

### Materials and Methods

Animals: Female 5-week-old golden Syrian hamsters were obtained from Charles River Laboratories (Wilmington, MA) for this experiment. The hamsters were quarantined for 3 days before use and maintained on Teklad Rodent Diet (Harlan Teklad) and tap water at the Laboratory Animal Research Center of Utah State University.

Virus: Severe Acute Respiratory Syndrome Coronavirus-2 (SARS-CoV-2) USA_WA1/2020 strain was obtained from the World Reference Center for Emerging Viruses and Arboviruses (WRCEVA). The virus was passaged two times in Vero 76 cells to generate a working stock for infection of hamsters.

Compounds: Berdazimer sodium was provided in a form ready to administer intranasally to animals. EIDD-2801 was purchased from MedChem Express by USU. EIDD-2801 (Molnupravir) is a ribonucleoside analog that has demonstrated activity across a wide range of RNA viruses including coronaviruses (2).

Experiment Design-Efficacy Study (PHA-254): A total of 55 hamsters were divided into 5 groups of 10 animals per treatment dose with 5 animals used as normal controls for weight gain (Table 2). For virus challenge, hamsters were anesthetized by IP injection of ketamine/xylazine (50 mg/kg/5 mg/kg) prior to challenge by the intranasal route with a dose of 1×10⁴³ 50% cell culture infectious doses (CCID₅₀) in a 100 μl inoculum volume. This 100 l total volume is delivered to both nostrils simultaneously. Treatment with berdazimer sodium was given once daily for seven days beginning 24 hours pre-infection by intranasal administration of 0.1 mL volume. All intranasal treatments were administered in a 100 μl volume after anesthetizing animals as was done for infections. Berdazimer sodium was prepared for dosing by preparing a stock in 100% dimethyl sulfoxide (DMSO) at 400, 200, and 100 mg/mL which was then diluted 200-fold into a buffer made up of a 1:1 ratio of Hyclone MEM from USU and 50 mM and pH 4.5 citrate. DMSO stocks of berdazimer sodium prepared 20-30 minutes prior to dosing were added to the MEM/citrate buffer within 5 minutes prior to dosing of animals. EIDD-2801 was administered per os (PO) twice daily beginning 4 hours post-infection. EIDD-2801 was solubilized in 10% DMSO and 90% corn oil. Five animals per group were euthanized on study days 3 and 6 to evaluate lung virus titers, nasal virus titers, and lung weights. Oropharyngeal swabs were collected from 5 animals per group on study days 1-6.

Experiment Design-Transmission Study (PHA-254B): A total of 20 5-week-old female golden Syrian hamsters were randomized into 2 groups of 2 animals and 4 groups of 4 animals (Table 3). Animals in groups 1 and 4 served as infected donor animals. Animals in group 2, 3, 5, and 6 were uninfected (naïve) animals and were cohabitated with animals from groups 1 or 4 for 4 hours each day on study days 1, 2, and 3. Animals in groups 1, 2, and 5 were treated with DMSO in MEM/citrate buffer as a placebo. Animals in groups 3, 4, and 6 were treated once daily with 2 mg/mL (2 mg/kg/d) berdazimer sodium 2 hours prior to cohabitation with infected animals. Treatment with berdazimer sodium began on the day of infection which was 24 hours prior to the first cohabitation session. Hamsters were weighed prior to infection and then everyday thereafter to evaluate infection-associated weight loss. All animals were euthanized on study day 4 to evaluate lung virus titers, nasal tissue virus titers, lung weights, and the transmission of virus from infected animals to naïve animals. Daily oropharyngeal swabs were collected on all animals.

Titration of Tissue and Oropharyngeal Swab Samples: Tissues homogenates and oropharyngeal swab samples were titrated by endpoint dilution. Serial log₁₀ dilutions of tissue homogenate or oropharyngeal swab samples were plated in quadruplicate wells of 96-well microplates containing confluent monolayers of Vero 76 cells. The plates were incubated in a 37° C. incubator with 5% CO2 for 6 days. The plates were then scored by visual observation under a light microscope for the presence of cytopathic effect (CPE). Virus titer for each sample was calculated by linear regression using the Reed-Muench method.

Statistics and Figures: Individual hamster body weights were converted to a percent of initial body weight on the day of infection. The percentage of initial body weight curves were compared using a one-way analysis of variance (ANOVA) comparing each treatment group to placebo-treated hamsters. Tissue virus titers and oropharyngeal swab samples compared using a two-way ANOVA comparing the treated animals to placebo-treated animals. Lung weight were compared using a one-way ANOVA since there was only one time point for evaluation.

Ethics regulation of laboratory animals: This study was conducted in accordance with the approval of the Institutional Animal Care and Use Committee of Utah State University dated Mar. 31, 2020 (expires Mar. 30, 2023). The work was done in the AAALAC-accredited Laboratory Animal Research Center of Utah State University. The U. S. Government (National Institutes of Health) approval was renewed Mar. 9, 2018 (PHS Assurance No. D16-00468[A3801-01]) in accordance to the National Institutes of Health Guide for the Care and Use of Laboratory Animals (Revision; 2011).

TABLE 2
Expt. PHA-254. Experimental Design-Efficacy Study

| Group | No. of | | | Treatment | |
|---|---|---|---|---|---|
| No. | Animals | Treatment | Dose | Schedule | Observations |
| 1 | 10 | Placebo | — | q.d. × 7 days | Five animals per group |
| 2 | 10 | Berdazimer Sodium | 0.5 mg/kg/d | beginning | euthanized on study |
| | | | (0.5 mg/mL | 24 hours | days 3 and 6 for |
| | | | solution) | pre-infection | evaluation of lung virus |
| 3 | 10 | Berdazimer Sodium | 1 mg/kg/d | | titers, lung weights, and |
| | | | (1 mg/mL | | nasal tissue titers. |
| | | | solution) | | Oropharyngeal swabs |
| 4 | 10 | Berdazimer Sodium | 2 mg/kg/d | | collected from 5 |
| | | | (2 mg/mL | | animals per group on |
| | | | solution) | | days 1-6. Animals |
| 5 | 10 | EIDD-2801 | 200 mg/kg/d | b.i.d. × 5 | weighed daily |
| | | | | days | throughout the study. |
| | | | | beginning 4 | |
| | | | | hours post- | |
| | | | | infection | |
| 5 | 5 | Normal Controls | — | — | Normal controls for |
| | | | | | weight gain. |

TABLE 3
Expt. PHA-254B. Experimental Design-Transmission Study

| Group | No. of | Infected | | Co-habitation | |
|---|---|---|---|---|---|
| No. | Animals | (Y/N) | Treatment | schedule | Observations |
| 1 | 2 | Y | Placebo-treated | N/A | All animals |
| | | | donor | | euthanized on |
| 2 | 4 | N | Placebo-treated- | Cohabitate with | study day 4 for |
| | | | naïve | group 1 animals-4 | evaluation of lung |
| 3 | 4 | N | Berdazimer | hours per day (days | virus titers, nasal |
| | | | sodium-treated- | 1-3). Animals split | tissue titers, and |
| | | | naïve | into two cages of 5 | lung weights. |
| | | | | animals each (one | Oropharyngeal |
| | | | | donor animal and two | swabs collected on |
| | | | | animals from each | days 1-4. |
| | | | | naïve group) | Animals weighed |
| 4 | 2 | Y | Berdazimer | N/A | daily throughout |
| | | | sodium-treated- | | the study. |
| | | | donor | | |
| 5 | 4 | N | Placebo-treated- | Cohabitate with | |
| | | | naïve | group 4 animals-4 | |
| 6 | 4 | N | Berdazimer | hours per day (days | |
| | | | sodium-treated- | 1-3). Animals split | |
| | | | naive | into two cages of 5 | |
| | | | | animals each (one | |
| | | | | donor animal and two | |
| | | | | animals from each | |
| | | | | naïve group) | |

### Results and Discussion

This study evaluated treatment with berdazimer sodium on the reduction of lung virus titers, nasal tissue titers, oropharyngeal swab titers, and lung weights in hamsters infected with SARS-CoV-2. In addition, the efficacy of intranasal treatment with berdazimer sodium on the transmission of SARS-CoV-2 in golden Syrian hamsters was evaluated.

In the efficacy study, a significant respiratory infection was observed as evidenced by high virus titers in the lung tissue, nasal tissue, and oropharyngeal swab titers. Lung virus titers on day 3 post-infection were approximately three logs higher than the inoculum dose indicating a robust infection in the hamster respiratory tissue. In addition, weight loss was observed in the placebo-treated animals.

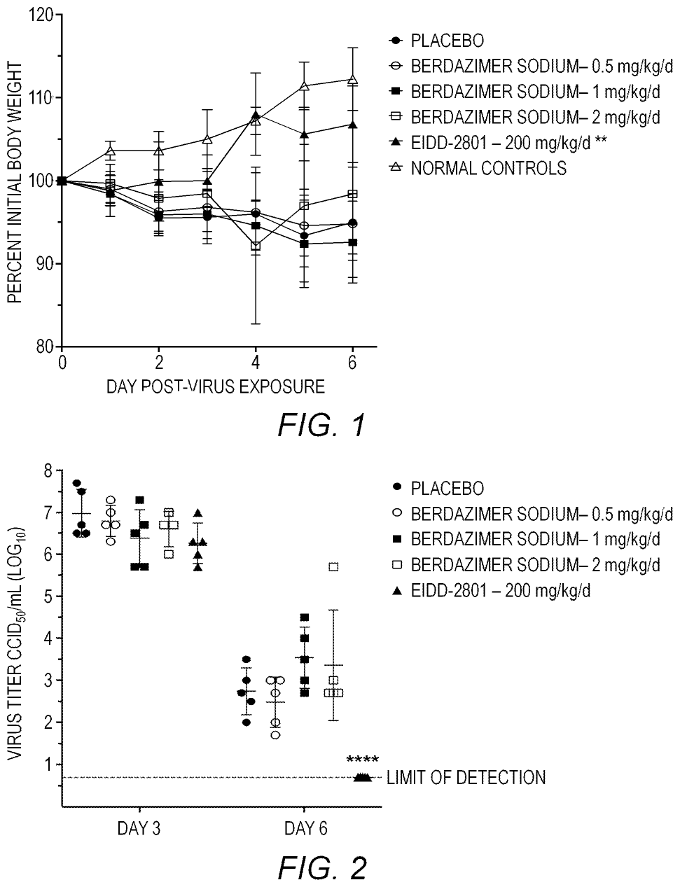

Percent initial body weight of 5-week-old golden Syrian hamsters following challenge with SARS-CoV-2 and treatment with berdazimer sodium is shown in FIG. 1. Treatment with berdazimer sodium did not prevent weight loss following infection. Treatment with EIDD-2801 at a dose of 200 mg/kg/d prevented weight loss in hamsters infected with SARS-CoV-2. Percent weight loss was evaluated using a one-way ANOVA comparing the average weight loss over the entire experiment for each treatment group.

FIG. 2 shows lung virus titers of 5-week-old golden Syrian hamsters after challenge with SARS-CoV-2 and treatment with berdazimer sodium. Treatment with berdazimer sodium did not significantly reduce lung virus titers in animals infected with SARS-CoV-2. Treatment with EIDD-2801 reduced lung virus titers of SARS-CoV-2-infected animals on day 6 post-infection. One animal in the group treated with 2 mg/kg/d of berdazimer sodium did not recover from the anesthesia following infection and was not included in the data set. Table 4 shows the lung and nasal virus titers on study days 3 and 6.

TABLE 4
Lung and nasal tissue virus titers on study days 3 and 6 of golden Syrian hamsters
after treatment with berdazimer sodium and infection with SARS-CoV-2.

| | | Virus Titer - *CCID₅₀/mL (Log₁₀) | | | | | | | | | |
|---|---|---|---|---|---|---|---|---|---|---|---|
| | | Day 3 | | | | | Day 6 | | | | |
| Treatment | Sample | 1 | 2 | 3 | 4 | 5 | 1 | 2 | 3 | 4 | 5 |
| Placebo | Lung tissue | 6.5 | 7.7 | 6.5 | 7.5 | 6.7 | 2.5 | 2.0 | 3.5 | 3.0 | 2.7 |
| Berdazimer sodium - 0.5 mg/kg/d | | 7.0 | 6.7 | 6.3 | 7.3 | 6.7 | 3.0 | 1.7 | 3.0 | 2.7 | 2.0 |
| Berdazimer sodium - 1 mg/kg/d | | 5.7 | 6.5 | 6.7 | 5.7 | 7.3 | 3.5 | 4.5 | 4.0 | 3.0 | 2.7 |
| Berdazimer sodium - 2 mg/kg/d | | 6.7 | 6.7 | 7.0 | 6.0 | X | 2.7 | 2.7 | 3.0 | 2.7 | 5.7 |
| EIDD-2801 - 200 mg/kg/d | | 6.3 | 6.0 | 5.7 | 7.0 | 6.3 | <0.7 | <0.7 | <0.7 | <0.7 | <0.7 |
| Placebo | Nasal tissue | 6.3 | 6.7 | 6.5 | 6.0 | 7.0 | <0.7 | <0.7 | <0.7 | 2.3 | 2.0 |
| Berdazimer sodium - 0.5 mg/kg/d | | 7.7 | 7.7 | 6.5 | 8.0 | 8.0 | 2.7 | 3.3 | <0.7 | 3.3 | 2.7 |
| Berdazimer sodium - 1 mg/kg/d | | 7.7 | 8.3 | 8.5 | 7.5 | 7.7 | 2.7 | 3.3 | <0.7 | 3.3 | 2.7 |
| Berdazimer sodium - 2 mg/kg/d | | 7.5 | 7.5 | 6.3 | 6.7 | X | 3.0 | <0.7 | 2.7 | 2.3 | 1.7 |
| EIDD-2801 - 200 mg/kg/d | | 7.5 | 6.7 | 7.5 | 6.5 | 7.0 | <0.7 | 3.3 | <0.7 | <0.7 | <0.7 |

*CCID₅₀—50% cell culture infectious dose
X—animal did not recover from anesthesia during infection

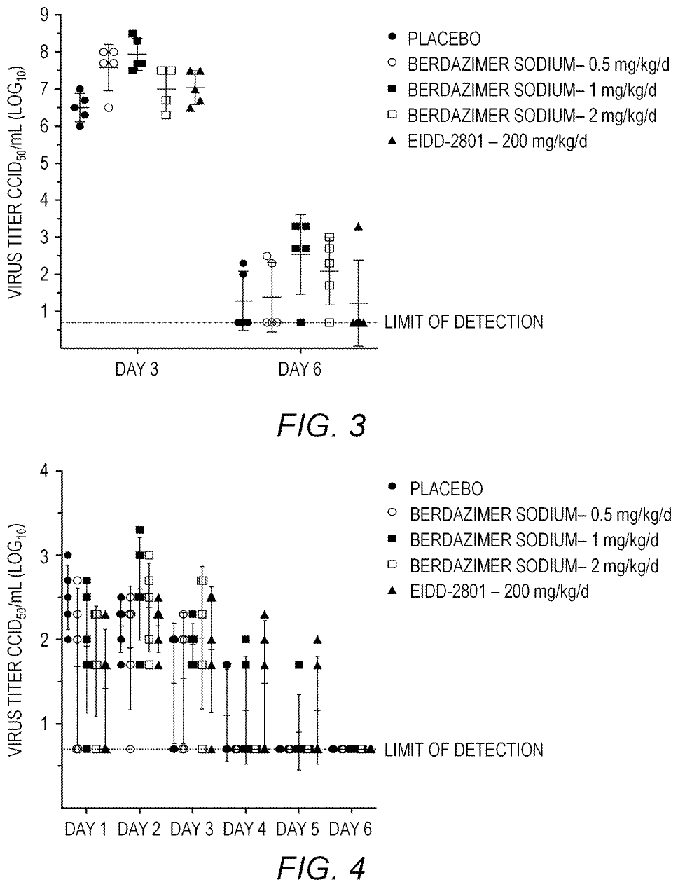

FIG. 3 shows nasal tissue virus titers of 5-week-old golden Syrian hamsters after challenge with SARS-CoV-2 and treatment with berdazimer sodium. Treatment with berdazimer sodium did not significantly reduce nasal tissue virus titers in animals infected with SARS-CoV-2. Treatment with EDD-2801 did not significantly reduce nasal virus titers after infection.

Oropharyngeal swab virus titers of 5-week-old golden Syrian hamsters after challenge with SARS-CoV-2 and treatment with berdazimer sodium are shown in FIG. 4. Treatment with berdazimer sodium did not reduce oropharyngeal swab virus titers in SARS-CoV-2-infected animals. EDD-2801 did not reduce oropharyngeal swab virus titers on any day post-infection. Table 5 shows the oropharyngeal swab virus titers on study days 1 through 6.

TABLE 5
Oropharyngeal swab virus titers on study days 1 through 6
of golden Syrian hamsters after treatment with berdazimer
sodium and infection with SARS-CoV-2-infected animals.

| | Day Post- | Virus Titer - *CCID₅₀/mL (Log₁₀) | | | | |
|---|---|---|---|---|---|---|
| Treatment | Infection | 1 | 2 | 3 | 4 | 5 |
| Placebo | 1 | 2.5 | 2.7 | 2.3 | 2.0 | 3.0 |
| Berdazimer sodium - 0.5 mg/kg/d | | <0.7 | <0.7 | 2.0 | 2.7 | 2.3 |
| Berdazimer sodium - 1 mg/kg/d | | 2.5 | 1.7 | 2.7 | <0.7 | 2.0 |
| Berdazimer sodium - 2 mg/kg/d | | 1.7 | 2.3 | 2.3 | 1.7 | <0.7 |
| EIDD-2801 - 200 mg/kg/d | | 2.3 | 1.7 | 1.7 | <0.7 | <0.7 |
| Placebo | 2 | 2.3 | 2.0 | 2.5 | 1.7 | 2.3 |
| Berdazimer sodium - 0.5 mg/kg/d | | 2.3 | <0.7 | 2.5 | 2.3 | 1.7 |
| Berdazimer sodium - 1 mg/kg/d | | 3.3 | 2.5 | 3.0 | 1.7 | 2.5 |
| Berdazimer sodium - 2 mg/kg/d | | 3.0 | 2.5 | 2.7 | 2.0 | 1.7 |
| EIDD-2801 - 200 mg/kg/d | | 2.3 | 1.7 | 2.5 | 2.3 | 2.0 |
| Placebo | 3 | 2.0 | <0.7 | 2.0 | <0.7 | 2.0 |
| Berdazimer sodium - 0.5 mg/kg/d | | 2.0 | <0.7 | 2.3 | 2.0 | <0.7 |
| Berdazimer sodium - 1 mg/kg/d | | 1.7 | 2.3 | 2.0 | 2.0 | 1.7 |
| Berdazimer sodium - 2 mg/kg/d | | 2.7 | 1.7 | <0.7 | 2.3 | 2.7 |
| EIDD-2801 - 200 mg/kg/d | | 2.5 | 1.7 | 2.5 | <0.7 | 2.0 |
| Placebo | 4 | <0.7 | <0.7 | 1.7 | <0.7 | 1.7 |
| Berdazimer sodium - 0.5 mg/kg/d | | <0.7 | <0.7 | <0.7 | <0.7 | <0.7 |
| Berdazimer sodium - 1 mg/kg/d | | 0.7 | 2.0 | 0.7 | 1.7 | 0.7 |
| Berdazimer sodium - 2 mg/kg/d | | <0.7 | <0.7 | <0.7 | <0.7 | <0.7 |
| EIDD-2801 - 200 mg/kg/d | | 2.3 | 1.7 | 2.0 | <0.7 | <0.7 |
| Placebo | 5 | <0.7 | <0.7 | <0.7 | <0.7 | <0.7 |
| Berdazimer sodium - 0.5 mg/kg/d | | <0.7 | <0.7 | <0.7 | <0.7 | <0.7 |
| Berdazimer sodium - 1 mg/kg/d | | <0.7 | 1.7 | <0.7 | <0.7 | <0.7 |
| Berdazimer sodium - 2 mg/kg/d | | <0.7 | <0.7 | <0.7 | <0.7 | <0.7 |
| EIDD-2801 - 200 mg/kg/d | | 2.0 | <0.7 | 1.7 | <0.7 | <0.7 |
| Placebo | 6 | <0.7 | <0.7 | <0.7 | <0.7 | <0.7 |
| Berdazimer sodium - 0.5 mg/kg/d | | <0.7 | <0.7 | <0.7 | <0.7 | <0.7 |
| Berdazimer sodium - 1 mg/kg/d | | <0.7 | <0.7 | <0.7 | <0.7 | <0.7 |
| Berdazimer sodium - 2 mg/kg/d | | <0.7 | <0.7 | <0.7 | <0.7 | <0.7 |
| EIDD-2801 - 200 mg/kg/d | | <0.7 | <0.7 | <0.7 | <0.7 | <0.7 |

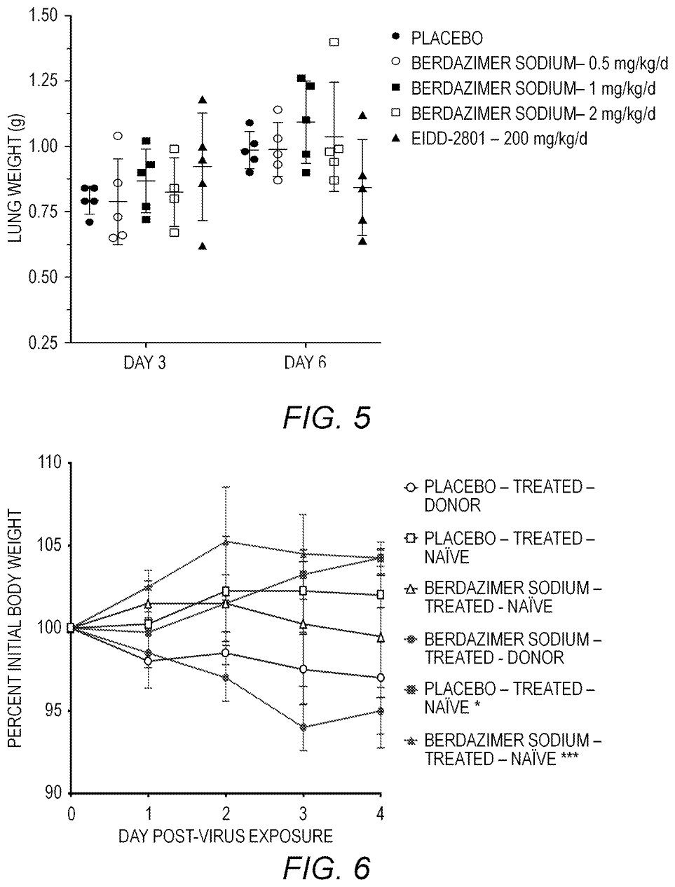

FIG. 5 shows lung weights of 5-week-old golden Syrian hamsters after challenge with SARS-CoV-2 and treatment with berdazimer sodium. Lung weights were not significantly affected by treatment with berdazimer sodium or EIDD-2801.

For the transmission study, cohabitation of donor animals with naïve animals produced a robust infection in all of the placebo-treated naïve animals as indicated by lung virus titers after cohabitation.

FIG. 6 shows percent initial body weight of 5-week-old golden Syrian hamsters treatment with berdazimer sodium and cohabitation with SARS-CoV-2-infected hamsters. Animals with the same color symbols were cohabitated on study days 1-3. Placebo-treated animals were protected from weight loss when the donor animals were treated with berdazimer sodium. Treatment with berdazimer sodium prevented weight loss following exposure to SARS-CoV-2-infected hamsters when the donor animals were also treated with berdazimer sodium. Percent weight loss was evaluated with a one-way ANOVA so weight loss for each treatment group was compared over the entire study.

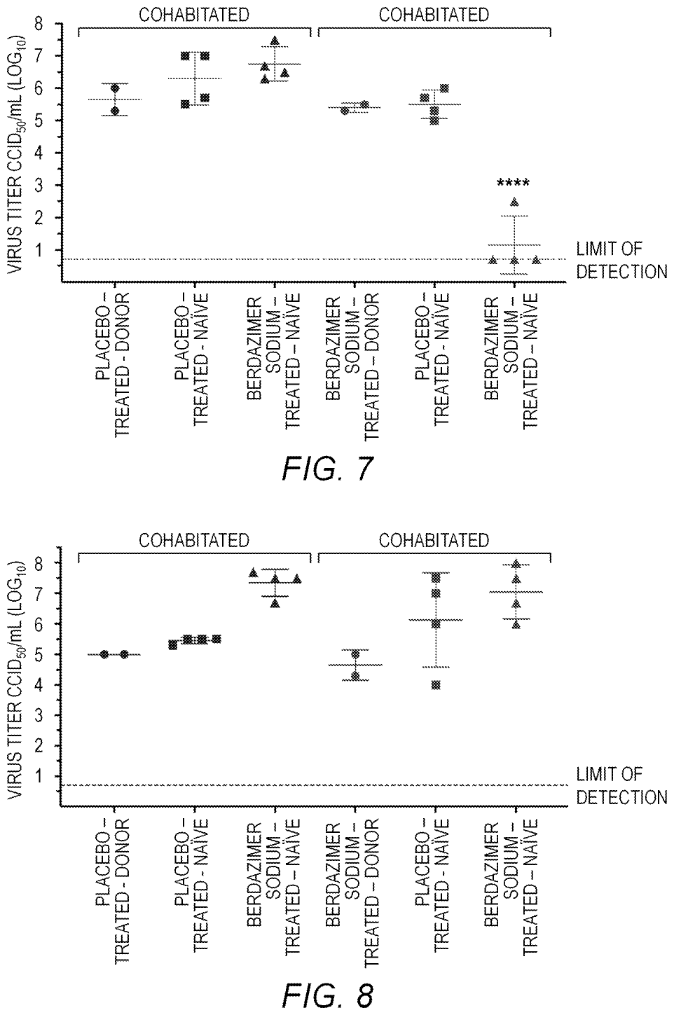

Lung virus titers of hamsters on day 4 treated with berdazimer sodium and exposed to SARS-CoV-2-infected hamsters are shown in FIG. 7. Animals that were cohabitated on study days 1-3 are denoted with brackets. Treatment with berdazimer sodium significantly reduced lung virus titers in treated naïve animals that were cohabitated with infected animals that were also treated with berdazimer sodium. Virus was only detected in one of four animals that were treated with berdazimer sodium and cohabitated with donor animals that were also treated with berdazimer sodium. The virus titer detected in the one animal was reduced by over two logs compared to naïve placebo-treated animals.

FIG. 8 shows nasal tissue virus titers of hamsters on day 4 treated with berdazimer sodium and exposed to SARS-CoV-2-infected hamsters. Animals that were cohabitated on study days 1-3 are denoted with brackets. Treatment with berdazimer sodium did not significantly reduce nasal tissue virus titers in animals cohabitated with SARS-CoV-2-infected hamsters. Table 6 shows lung and nasal tissue virus titers of golden Syrian hamsters after treatment with berdazimer sodium prior to cohabitation with SARS-CoV-2-infected animals.

TABLE 6
Lung and nasal tissue virus titers of golden Syrian hamsters after treatment with
berdazimer sodium prior to cohabitation with SARS-COV-2-infected animals.

| | | Virus Titer-*CCID₅₀/mL (Log₁₀) | | | |
|---|---|---|---|---|---|
| | | Study Day 4 | | | |
| Treatment | Sample | 1 | 2 | 3 | 4 |
| Group 1-Placebo-treated-donor | Lung tissue | 5.3 | 6.0 | — | — |
| Group 2-Placebo-treated-naïve | | 5.5 | 7.0 | 5.7 | 7.0 |
| Group 3-Berdazimer-sodium- | | 7.5 | 6.3 | 6.7 | 6.5 |
| treated-naïve | | | | | |
| Group 4-Berdazimer sodium- | | 5.3 | 5.5 | — | — |
| treated-donor | | | | | |
| Group 5-Placebo-treated-naïve | | 6.0 | 5.0 | 5.3 | 5.7 |
| Group 6-Berdazimer sodium- | | 2.5 | <0.7 | <0.7 | <0.7 |
| treated-naive | | | | | |
| Group 1-Placebo-treated-donor | Nasal tissue | 5.0 | 5.0 | — | — |
| Group 2-Placebo-treated-naïve | | 5.3 | 5.5 | 5.5 | 5.5 |
| Group 3-Berdazimer sodium- | | 6.7 | 7.5 | 7.7 | 7.5 |
| treated-naïve | | | | | |
| Group 4-Berdazimer sodium- | | 4.3 | 5.0 | — | — |
| treated-donor | | | | | |
| Group 5-Placebo-treated-naïve | | 7.0 | 7.5 | 6.0 | 4.0 |
| Group 6-Berdazimer sodium- | | 8.0 | 7.5 | 6.7 | 6.0 |
| treated-naive | | | | | |

*CCID₅₀-50% cell culture infectious dose

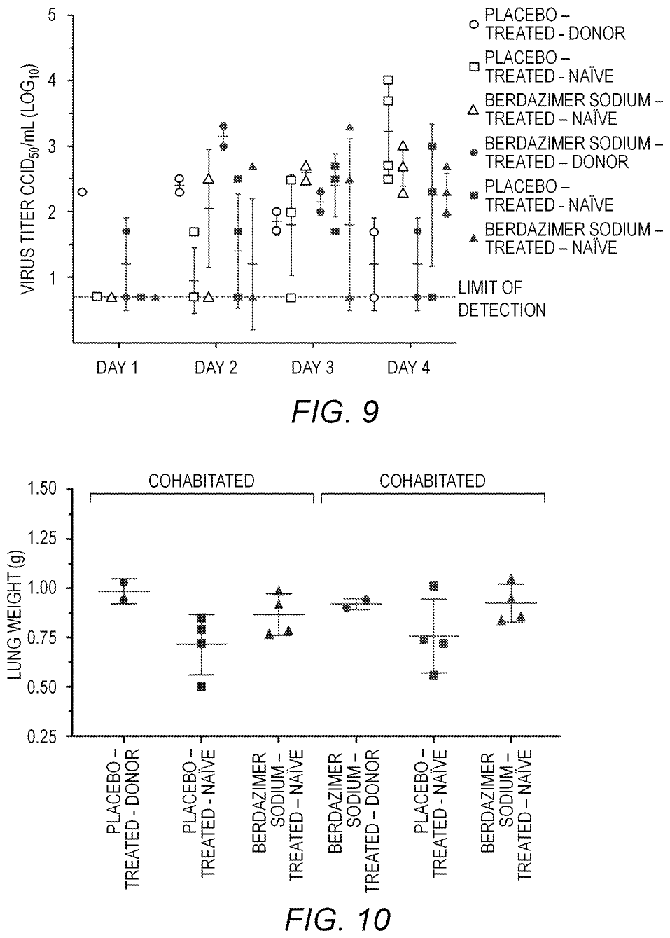

FIG. 9 shows oropharyngeal swab virus titers of 5-week-old golden Syrian hamsters aftertreatment with berdazimer sodium and cohabitation with SARS-CoV-2-infected hamsters. Treatment with berdazimer sodium at a dose of 2 mg/kg/d did not significantly reduce oropharyngeal swab titers. Table 7 shows oropharyngeal swab virus titers on study days 1 through 4 of golden Syrian hamsters after treatment with berdazimer sodium prior to cohabitation with SARS-CoV-2-infected animals.

TABLE 7
Oropharyngeal swab virus titers on study days 1 through 4 of golden Syrian
hamsters after treatment with berdazimer sodium prior to cohabitation with
SARS-COV-2-infected animals.

| | | Virus Titer-*CCID₅₀/mL (Log₁₀) | | | |
|---|---|---|---|---|---|
| Treatment | Study Day | 1 | 2 | 3 | 4 |
| Group 1-Placebo-treated-donor | 1 | 2.3 | 2.3 | — | — |
| Group 2-Placebo-treated-naïve | | <0.7 | <0.7 | <0.7 | <0.7 |
| Group 3-Berdazimer sodium | | <0.7 | <0.7 | <0.7 | <0.7 |
| Treated-naïve | | | | | |
| Group 4-Berdazimer sodium | | 1.0 | <0.7 | — | — |
| Treated-donor | | | | | |
| Group 5-Placebo-treated-naïve | | <0.7 | <0.7 | <0.7 | <0.7 |
| Group 6-Berdazimer sodium- | | <0.7 | <0.7 | <0.7 | <0.7 |
| treated-naive | | | | | |
| Group 1-Placebo-treated-donor | 2 | 2.3 | 2.5 | — | — |
| Group 2-Placebo-treated-naïve | | 1.7 | <0.7 | <0.7 | <0.7 |
| Group 3-Berdazimer sodium | | 2.5 | 2.5 | 2.5 | <0.7 |
| Treated-naïve | | | | | |
| Group 4-Berdazimer sodium | | 3.0 | 3.3 | — | — |
| Treated-donor | | | | | |
| Group 5-Placebo-treated-naïve | | 1.7 | <0.7 | 2.5 | <0.7 |
| Group 6-Berdazimer sodium- | | <0.7 | 2.7 | <0.7 | <0.7 |
| treated-naive | | | | | |
| Group 1-Placebo-treated-donor | 3 | 1.7 | 2.0 | — | — |
| Group 2-Placebo-treated-naïve | | 2.0 | 2.5 | <0.7 | 2.0 |
| Group 3-Berdazimer sodium | | 2.5 | 2.7 | 2.7 | 2.5 |
| Treated-naïve | | | | | |
| Group 4-Berdazimer sodium | | 2.3 | 2.0 | — | — |
| Treated-donor | | | | | |
| Group 5-Placebo-treated-naïve | | 2.5 | 1.7 | 2.7 | 2.7 |
| Group 6-Berdazimer sodium- | | <0.7 | 3.3 | 2.5 | <0.7 |
| treated-naive | | | | | |
| Group 1-Placebo-treated-donor | 4 | <0.7 | 1.7 | — | — |
| Group 2-Placebo-treated-naïve | | 2.7 | 4.0 | 2.5 | 3.7 |
| Group 3-Berdazimer sodium | | 2.3 | 3.0 | 2.7 | 2.7 |
| Treated-naïve | | | | | |
| Group 4-Berdazimer sodium | | <0.7 | 1.7 | — | — |
| Treated-donor | | | | | |
| Group 5-Placebo-treated-naïve | | 2.3 | 3.0 | 3.0 | <0.7 |
| Group 6-Berdazimer sodium- | | 2.0 | 2.3 | 2.7 | 2.0 |
| treated-naive | | | | | |

*CCID₅₀-50% cell culture infectious dose

Lung weights of 5-week-old golden Syrian hamsters after treatment with berdazimer sodium and cohabitation with SARS-CoV-2-infected animals are shown in FIG. 10. Lung weights were not statistically different between groups when compared by one-way ANOVA.

### Conclusions

This study evaluated the effect of treatment with berdazimer sodium on weight loss, lung virus titers, nasal tissue titers, oropharyngeal swab titers, and lung weights. The ability of berdazimer sodium treatment to prevent transmission of SARS-CoV-2 to naïve animals was also evaluated.

In the efficacy study, treatment with berdazimer sodium did not prevent weight loss or reduce lung virus titers, nasal tissue titers, or oropharyngeal swab titers at any dose.

However, treatment with berdazimer sodium at a dose of 2 mg/kg/d significantly reduced lung virus burden of SARS-CoV-2 in naïve animals when the donor animals were also treated with 2 mg/mL berdazimer sodium. The virus was not detected in the lungs of three of the four naïve animals that were exposed to infected animals and was reduced by two logs in the remaining animal. Virus was detected in the nasal tissue samples of all four animals indicating that the animals were infected but that treatment with berdazimer sodium prevented the virus from infecting the lungs. This is also supported by the percent initial body weight data that shows that the same animals were protected from weight loss after exposure to SARS-CoV-2-infected animals. Oropharyngeal swab titers and lung weights were not affected by berdazimer sodium treatment in the transmission study.

It is interesting to note that berdazimer sodium treatment was able to decrease transmission of SARS-CoV-2 from infected animals even though no effect was observed in the efficacy study. No adverse events were observed in the hamsters treated with 2 mg/kg/d of berdazimer sodium.

### Example 3

The objective of this study was to confirm the efficacy of berdazimer sodium at reducing the direct transmission of SARS-CoV-2 from infected wild-type golden Syrian hamsters to naïve littermates.

The effect of berdazimer sodium treatment on weight loss, lung virus titers, nasal tissue titers, and lung weights in hamsters exposed to SARS-CoV-2 were the primary endpoints.

### Materials and Methods

Animals: Female 16-week-old golden Syrian hamsters were obtained from a colony maintained at Utah State University for this experiment. The hamsters were quarantined for 3 days before use and maintained on Teklad Rodent Diet (Harlan Teklad) and tap water at the Laboratory Animal Research Center of Utah State University.

Virus: Severe Acute Respiratory Syndrome Coronavirus-2 (SARS-CoV-2) USA_WA1/2020 strain was obtained from the World Reference Center for Emerging Viruses and Arboviruses (WRCEVA). The virus was passaged two times in Vero 76 cells to generate a working stock for infection of hamsters.

Compounds: Berdazimer sodium was provided as a solid and was solubilized in DMSO prior to treatment. The DMSO stock was added to a 1:1 mixture of minimum essential media (MEM) and 50 mM, pH 4.5 citrate buffer.

Experiment Design-Transmission Study: A total of 43 16-week-old female golden Syrian hamsters were randomized into 4 groups of 2 animals and 8 groups of 4 animals with 3 animals retained as normal controls (Table 8). Animals in groups 1, 4, 7, and 10 served as infected donor animals. Animals in group 2, 3, 5, 6, 8, 9, 11, and 12 were uninfected (naïve) animals and were cohabitated with animals from groups 1, 4, 7, or 10 for 4 hours each day on study days 1, 2, and 3. Animals in groups 1, 2, 5, 8, and 11 were treated with DMSO in MEM/citrate buffer as a placebo. Animals in groups 3, 4, 6, 7, 9, 10, and 12 were treated once daily with either, 1, 2, or 8 mg/mL (1,2, or 8 mg/kg/d) berdazimer sodium 2 hours prior to cohabitation with infected animals. For infected donor animals, treatment with berdazimer sodium began on the day of infection which was 24 hours prior to the first cohabitation session. Hamsters were weighed prior to infection and then everyday thereafter to evaluate infection-associated weight loss. All animals were euthanized on study day 4 to evaluate lung virus titers, nasal tissue virus titers, lung weights, and the transmission of virus from infected animals to naïve animals.

TABLE 8
Experimental design.

| Group | No. of | Infected | | | |
|---|---|---|---|---|---|
| No. | Animals | (Y/N) | Treatment | Co-habitation schedule | Observations |
| 1 | 2 | Y | Placebo-treated | N/A | All animals |
| | | | donor | | euthanized on |
| 2 | 4 | N | Placebo-treated- | Cohabitate with group 1 | study day 4 |
| | | | naïve | animals-4 hours per day | for evaluation |
| 3 | 4 | N | Berdazimer | (days 1-3). Animals split | of lung virus |
| | | | sodium-treated | into two cages of 5 animals | titers, nasal |
| | | | (8 mg/ml)-naïve | each (one donor animal | tissue titers, |
| | | | | and two animals from each | and lung |
| | | | | naïve group) | weights. |
| 4 | 2 | Y | Berdazimer | N/A | Animals |
| | | | sodium-treated | | weighed daily |
| | | | (8 mg/ml)-donor | | throughout |
| 5 | 4 | N | Placebo-treated- | Cohabitate with group 4 | the study. |
| | | | naïve | animals-4 hours per day | |
| 6 | 4 | N | Berdazimer | (days 1-3). Animals split | |
| | | | sodium-treated | into two cages of 5 animals | |
| | | | (8 mg/ml)-naïve | each (one donor animal | |
| | | | | and two animals from each | |
| | | | | naïve group) | |
| 7 | 2 | Y | Berdazimer | N/A | |
| | | | sodium-treated | | |
| | | | (2 mg/ml)-donor | | |
| 8 | 4 | N | Placebo-treated- | Cohabitate with group 7 | |
| | | | naïve | animals-4 hours per day | |
| | | | | (days 1-3). Animals split | |
| 9 | 4 | N | Berdazimer | into two cages of 5 animals | |
| | | | sodium-treated | each (one donor animal | |
| | | | (2 mg/ml)-naïve | and two animals from each | |
| | | | | naïve group) | |
| 10 | 2 | Y | Berdazimer | N/A | |
| | | | sodium-treated | | |
| | | | (1 mg/ml)-donor | | |
| 11 | 4 | N | Placebo-treated- | Cohabitate with group 10 | |
| | | | naïve | animals-4 hours per day | |
| 12 | 4 | N | Berdazimer | (days 1-3). Animals split | |
| | | | sodium-treated | into two cages of 5 animals | |
| | | | (1 mg/ml)-naive | each (one donor animal | |
| | | | | and two animals from each | |
| | | | | naïve group) | |
| 13 | 3 | N | Normal controls | NA | Observed for |
| | | | | | weight gain |

Titration of Tissue Samples: Tissues homogenates were titrated by endpoint dilution. Serial log₁₀ dilutions of tissue homogenates were plated in quadruplicate wells of 96-well microplates containing confluent monolayers of Vero 76 cells. The plates were incubated in a 37° C. incubator with 5% CO2 for 6 days. The plates were then scored by visual observation under a light microscope for the presence of cytopathic effect (CPE). Virus titer for each sample was calculated by linear regression using the Reed-Muench method.

Statistics and Figures: Individual hamster body weights were converted to a percent of initial body weight on the day of infection. The percentage of initial body weight curves were compared using a one-way analysis of variance (ANOVA) comparing each treatment group to placebo-treated hamsters. Tissue virus titers and lung weights were compared using a one-way ANOVA comparing the treated animals to placebo-treated animals.

Ethics regulation of laboratory animals: This study was conducted in accordance with the approval of the Institutional Animal Care and Use Committee of Utah State University dated Mar. 31, 2020 (expires Mar. 30, 2023). The work was done in the AAALAC-accredited Laboratory Animal Research Center of Utah State University. The U. S. Government (National Institutes of Health) approval was renewed Mar. 9, 2018 (PHS Assurance No. D16-00468[A3801-01]) in accordance to the National Institutes of Health Guide for the Care and Use of Laboratory Animals (Revision; 2011).

### Results and Discussion

This study evaluated the efficacy of intranasal treatment with berdazimer sodium on the transmission of SARS-CoV-2 in golden Syrian hamsters.

For the transmission study, cohabitation of donor animals with naïve animals produced a robust infection in all of the placebo-treated naïve animals as indicated by lung virus titers after cohabitation.

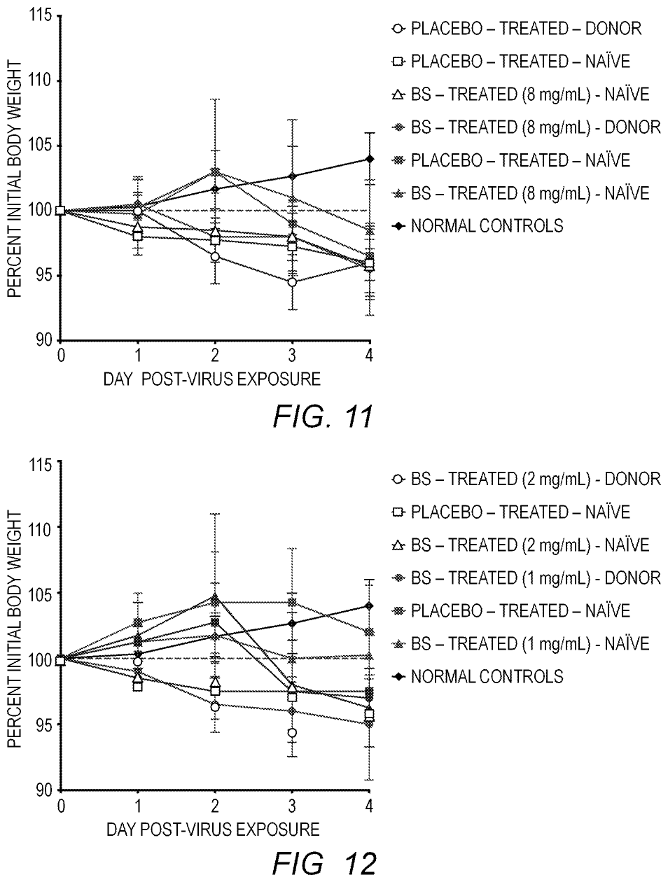

FIG. 11 shows percent initial body weight of 16-week-old golden Syrian hamsters treatment with berdazimer sodium (8 mg/ml) and cohabitation with SARS-CoV-2-infected hamsters. Animals with the same color symbols were cohabitated on study days 1-3. No significant differences in body weight were observed in hamsters treated with berdazimer sodium (8 mg/ml) and cohabitated with SARS-CoV-2-infected hamsters.

FIG. 12 shows percent initial body weight of 16-week-old golden Syrian hamsters after treatment with berdazimer sodium (2 or 1 mg/ml) and cohabitation with SARS-CoV-2-infected hamsters. Animals with the same color symbols were cohabitated on study days 1-3. No significant differences in body weight were observed in hamsters treated with berdazimer sodium (2 or 1 mg/ml) and cohabitated with SARS-CoV-2-infected hamsters.

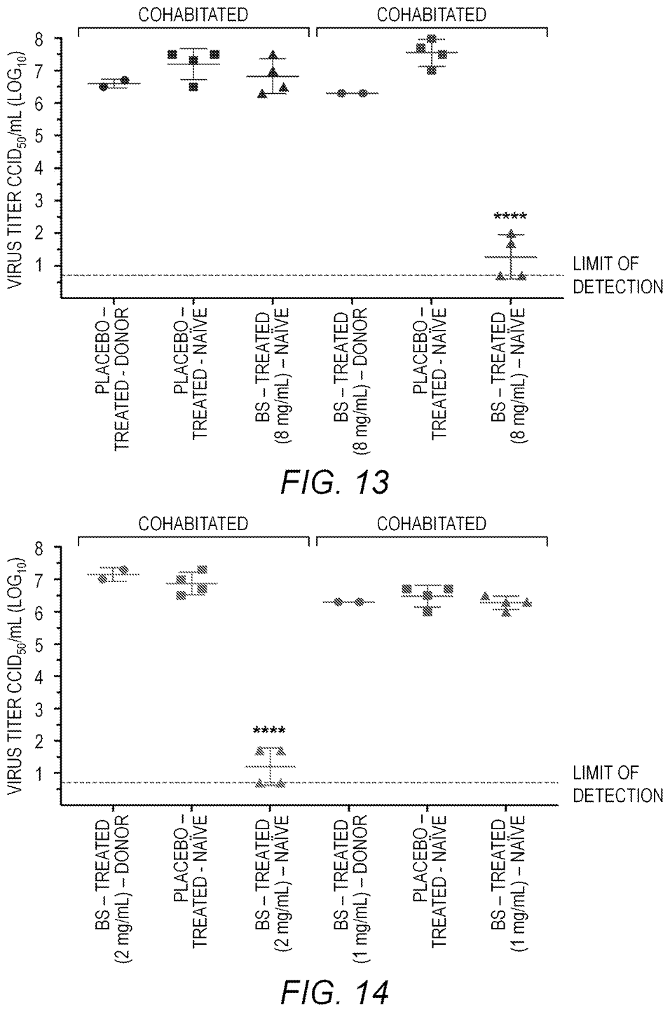

Lung virus titers of hamsters on day 4 treated with berdazimer sodium (8 mg/ml) and exposed to SARS-CoV-2-infected hamsters are shown in FIG. 13. Animals that were cohabitated on study days 1-3 are denoted with brackets. Treatment with berdazimer sodium (8 mg/ml) significantly reduced lung virus titers in treated naïve animals that were cohabitated with infected animals that were also treated with berdazimer sodium (8 mg/ml). Virus was only detected in two of four animals that were treated with berdazimer sodium (8 mg/ml) and cohabitated with donor animals that were also treated with berdazimer sodium (8 mg/ml). The virus titers detected in the two animals were reduced by over five logs compared to naïve placebo-treated animals.

Lung virus titers of hamsters on day 4 treated with berdazimer sodium (2 or 1 mg/ml) and exposed to SARS-CoV-2-infected hamsters are shown in FIG. 14. Animals that were cohabitated on study days 1-3 are denoted with brackets. Treatment with berdazimer sodium (2 mg/ml) significantly reduced lung virus titers in treated naïve animals that were cohabitated with infected animals that were also treated with berdazimer sodium (2 mg/ml). Virus was only detected in two of four animals that were treated with berdazimer sodium (2 mg/ml) and cohabitated with donor animals that were also treated with berdazimer sodium (2 mg/ml). The virus titers detected in the two animals were reduced by over five logs compared to naïve placebo-treated animals. Treatment with berdazimer sodium (1 mg/ml) did not significantly reduce lung virus titers in treated naïve animals that were cohabitated with infected animals that were also treated with berdazimer sodium (1 mg/ml). Table 9 shows Lung virus titers of golden Syrian hamsters after treatment with berdazimer sodium prior to cohabitation with SARS-CoV-2-infected animals.

TABLE 9
Lung virus titers of golden Syrian hamsters after treatment with berdazimer
sodium prior to cohabitation with SARS-CoV-2-infected animals.

| | | Virus Titer-*CCID₅₀/mL (Log10) | | | |
|---|---|---|---|---|---|
| | | Study Day 4 | | | |
| Treatment | Sample | 1 | 2 | 3 | 4 |
| Group 1-Placebo-treated-donor | Lung tissue | 6.5 | 6.7 | — | — |
| Group 2-Placebo-treated-naïve | | 7.5 | 7.3 | 6.5 | 7.5 |
| Group 3-BS-treated (8 mg/ml)-naïve | | 7.0 | 7.5 | 6.3 | 6.5 |
| Group 4-BS-treated (8 mg/ml)-donor | | 6.3 | 6.3 | — | — |
| Group 5-Placebo-treated-naïve | | 7.7 | 7.0 | 7.5 | 8.0 |
| Group 6-BS-treated (8 mg/ml)-naïve | | <0.7 | 1.7 | <0.7 | 2.0 |
| Group 7-BS-treated (2 mg/ml)-donor | | 7.3 | 7.0 | — | — |
| Group 8-Placebo-treated-naïve | | 6.7 | 7.0 | 6.5 | 7.3 |
| Group 9-BS-treated (2 mg/ml)-naïve | | 1.7 | <0.7 | 1.7 | <0.7 |
| Group 10-BS-treated (1 mg/ml)-donor | | 6.3 | 6.3 | — | — |
| Group 11-Placebo-treated-naïve | | 6.0 | 6.7 | 6.5 | 6.7 |
| Group 12-BS-treated (1 mg/ml)-naïve | | 6.3 | 6.5 | 6.3 | 6.0 |

*CCID₅₀-50% cell culture infectious dose

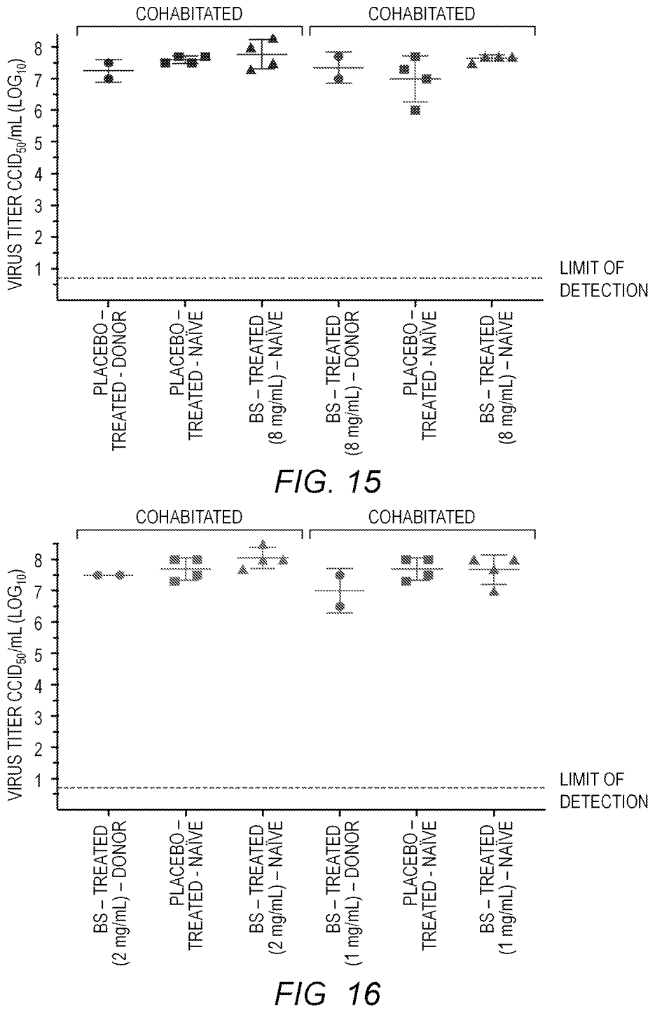

FIG. 15 shows nasal tissue virus titers of hamsters on day 4 treated with berdazimer sodium (8 mg/ml) and exposed to SARS-CoV-2-infected hamsters. Animals that were cohabitated on study days 1-3 are denoted with brackets. Treatment with berdazimer sodium (8 mg/ml) did not significantly reduce nasal tissue virus titers in animals cohabitated with SARS-CoV-2-infected hamsters.

FIG. 16 shows nasal tissue virus titers of hamsters on day 4 treated with berdazimer sodium (2 or 1 mg/ml) and exposed to SARS-CoV-2-infected hamsters. Animals that were cohabitated on study days 1-3 are denoted with brackets. Treatment with berdazimer sodium (2 or 1 mg/ml) did not significantly reduce nasal tissue virus titers in animals cohabitated with SARS-CoV-2-infected hamsters. Table 10 shows the nasal tissue virus titers of golden Syrian hamsters after treatment with berdazimer sodium prior to cohabitation with SARS-CoV-2-infected animals.

TABLE 10
Nasal tissue virus titers of golden Syrian hamsters after treatment with berdazimer
sodium prior to cohabitation withSARS-COV-2-infected animals.

| | | Virus Titer-*CCID₅₀/mL (Log10) | | | |
|---|---|---|---|---|---|
| | | Study Day 4 | | | |
| Treatment | Sample | 1 | 2 | 3 | 4 |
| Group 1-Placebo-treated-donor | Nasal tissue | 7.0 | 7.5 | — | — |
| Group 2-Placebo-treated-naïve | | 7.5 | 7.5 | 7.7 | 7.7 |
| Group 3-BS-treated (8 mg/ml)-naïve | | 8.0 | 7.5 | 7.3 | 8.3 |
| Group 4-BS-treated (8 mg/ml)-donor | | 7.7 | 7.0 | — | — |
| Group 5-Placebo-treated-naïve | | 7.3 | 7.7 | 6.0 | 7.0 |
| Group 6-BS-treated (8 mg/ml)-naïve | | 7.7 | 7.7 | 7.7 | 7.5 |
| Group 7-BS-treated (2 mg/ml)-donor | | 7.5 | 7.5 | — | — |
| Group 8-Placebo-treated-naïve | | 7.5 | 8.0 | 8.0 | 7.7 |
| Group 9-BS-treated (2 mg/ml)-naïve | | 8.5 | 8.0 | 8.0 | 7.7 |
| Group 10-BS-treated (1 mg/ml)-donor | | 6.5 | 7.5 | — | — |
| Group 11-Placebo-treated-naïve | | 8.0 | 7.5 | 8.0 | 7.3 |
| Group 12-BS-treated (1 mg/ml)-naïve | | 7.0 | 7.7 | 8.0 | 8.0 |

*CCID₅₀-50% cell culture infectious dose

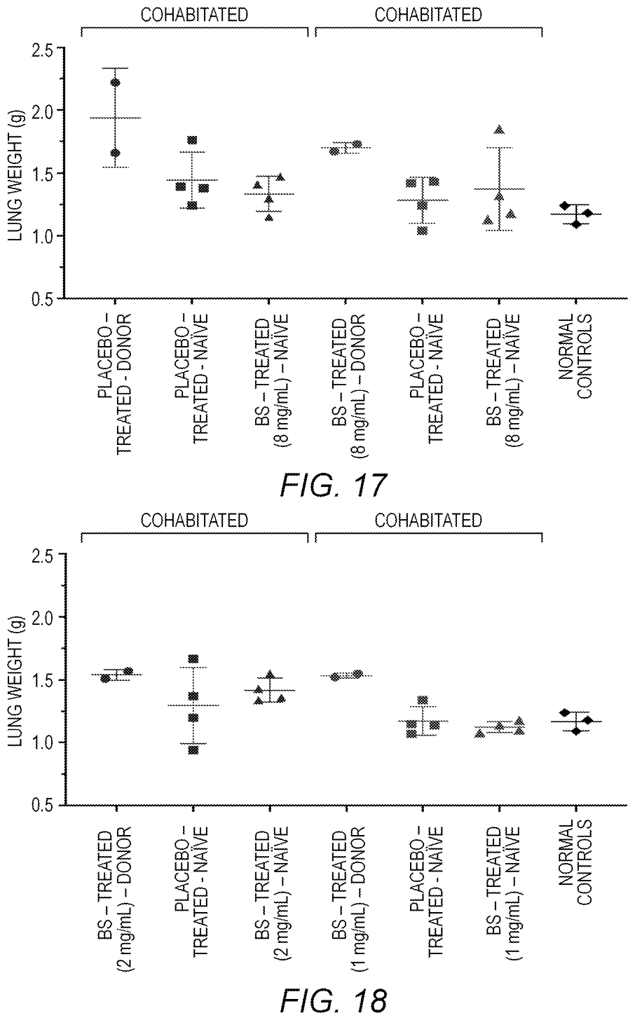

Lung weights of 16-week-old golden Syrian hamsters after treatment with berdazimer sodium (8 mg/ml) and cohabitation with SARS-CoV-2-infected animals are shown in FIG. 17. Lung weights were not statistically different between groups when compared by one-way ANOVA.

Lung weights of 16-week-old golden Syrian hamsters after treatment with berdazimer sodium (2 or 1 mg/ml) and cohabitation with SARS-CoV-2-infected animals are shown in FIG. 18. Lung weights were not statistically different between groups when compared by one-way ANOVA.

### Conclusions

This study evaluated the ability of berdazimer sodium treatment to prevent transmission of SARS-CoV-2 to naïve animals.

Treatment with berdazimer sodium at doses of 8 or 2 mg/ml significantly reduced lung virus burden of SARS-CoV-2 in naïve animals when the donor animals were also treated with 8 or 2 mg/mL berdazimer sodium. In each dose group, the virus was not detected in the lungs of two of the four naïve animals that were exposed to infected animals and was reduced by five logs in the remaining animals. Virus was detected in the nasal tissue samples of all four animals per dose group indicating that the animals were infected but that treatment with berdazimer sodium prevented the virus from infecting the lungs. The lack of a positive benefit from the 1 mg/mL treatment illustrates a dose response for berdazimer sodium in this study.

These lung viral titer findings confirm the results observed for 2 mg/mL in Example 2. However, an improvement in weight loss was not observed in this study whereas it was observed in Example 2. This may be due to the older age of hamsters that were used in this current study.

### Example 4

The objective of this study was to assess the efficacy of berdazimer sodium for treatment of a SARS-CoV-2 infection in wild-type golden Syrian hamsters. Administration of berdazimer sodium in either daily or twice daily treatment regimens at a 2 mg/mL treatment dose and in daily treatment regimens at 4 and 8 mg/mL treatment doses were evaluated.

The effect of berdazimer sodium treatment on weight loss, lung virus titers, nasal tissue titers, and lung weights in hamsters exposed to SARS-CoV-2 were the primary endpoints.

### Materials and Methods

Animals: Female 5-week-old golden Syrian hamsters were obtained from Charles River Laboratories (Wilmington, MA) for this experiment. The hamsters were quarantined for 3 days before use and maintained on Teklad Rodent Diet (Harlan Teklad) and tap water at the Laboratory Animal Research Center of Utah State University.

Virus: Severe Acute Respiratory Syndrome Coronavirus-2 (SARS-CoV-2) USA_WA1/2020 strain was obtained from the World Reference Center for Emerging Viruses and Arboviruses (WRCEVA). The virus was passaged two times in Vero 76 cells to generate a working stock for infection of hamsters.

Compounds: Berdazimer sodium was provided in form ready to administer intranasally to animals. EIDD-2801 was purchased from MedChem Express by USU. EIDD-2801 (Molnupravir) is a ribonucleoside analog that has demonstrated activity across a wide range of RNA viruses including coronaviruses.

Experiment Design-Efficacy Study (PHA-262): A total of 85 hamsters were divided into 5 groups of 10 animals per once daily treatment dose and 2 groups of 15 animals per twice daily treatment dose (Table 11). For virus challenge, hamsters were anesthetized by IP injection of ketamine/xylazine (50 mg/kg/5 mg/kg) prior to challenge by the intranasal route with a dose of 1×10⁴ 50% cell culture infectious doses (CCID₅₀) in a 100 μl inoculum volume. This 100 μl total volume is delivered to both nostrils simultaneously. Treatment with berdazimer sodium was given once or twice daily for seven days beginning 24 hours pre-infection by intranasal administration of 0.1 mL volume. Twice daily doses were administered 12 hours apart. All intranasal treatments were administered in a 100 μl volume after anesthetizing animals as was done for infections. Berdazimer sodium was prepared for dosing by first preparing a stock. Briefly, stock was prepared in 100% dimethylsfuloxide (DMSO) at 1600, 800, and 400 mg/mL which was then diluted 200-fold into a buffer made up of a 1:1 ratio of Hyclone MEM from USU and 50 mM and pH 4.5 citrate. DMSO stocks of berdazimer sodium were prepared 20-30 minutes prior to dosing and were added to the MEM/citrate buffer within 5 minutes prior to dosing of animals. EIDD-2801 was administered per os (PO) twice daily beginning 4 hours post-infection. EIDD-2801 was solubilized in 10% DMSO and 90% corn oil. Five animals per group were euthanized on study days 3 and 6 to evaluate lung virus titers, nasal virus titers, and lung weights. In groups 1 and 2, 10 animals from each group were euthanized on day 6 since 5 extra animals were included in these groups in the event that they did not recover from anesthesia.

TABLE 11
Experimental Design-Efficacy Study

| Group | No. of | | | Treatment | |
|---|---|---|---|---|---|
| No. | Animals | Treatment | Dose | Schedule | Observations |
| 1 | 15 | Placebo | — | b.i.d. × 7 | Five animals per |
| 2 | 15 | Berdazimer Sodium | 2 mg/kg/dose | days | group on study day 3 |
| | | | (2 mg/mL | beginning 24 | and 10 animals on |
| | | | solution) | hours pre- | study day 6 were |
| | | | | infection | euthanized for |
| | | | | | evaluation of lung |
| | | | | | virus titers, lung |
| | | | | | weights, and nasal |
| | | | | | tissue titers. Animals |
| | | | | | weighed daily |
| | | | | | throughout the study. |
| 3 | 10 | Placebo | — | q.d. × 7 days | Five animals per |
| 4 | 10 | Berdazimer Sodium | 2 mg/kg/dose | beginning 24 | group euthanized on |
| | | | (2 mg/mL | hours pre- | study days 3 and 6 for |
| | | | solution) | infection | evaluation of lung |
| 5 | 10 | Berdazimer Sodium | 4 mg/kg/dose | | virus titers, lung |
| | | | (4 mg/mL | | weights, and nasal |
| | | | solution) | | tissue titers. Animals |
| 6 | 10 | Berdazimer Sodium | 8 mg/kg/dose | | weighed daily |
| | | | (8 mg/mL | | throughout the study. |
| | | | solution) | | |
| 7 | 10 | EIDD-2801 | 500 mg/kg/day | b.i.d. × 5 | |
| | | | | days | |
| | | | | beginning 4 | |
| | | | | hours post- | |
| | | | | infection | |

Titration of Tissue Samples: Lung and nasal tissue homogenates were titrated by endpoint dilution. Serial log₁₀ dilutions of tissue homogenate were plated in quadruplicate wells of 96-well microplates containing confluent monolayers of Vero 76 cells. The plates were incubated in a 37° C. incubator with 500 CO2 for 6 days. The plates were then scored by visual observation under a light microscope for the presence of cytopathic effect (CPE). Virus titer for each sample was calculated by linear regression using the Reed-Muench method.

Statistics and Figures: Individual hamster body weights were converted to a percent of initial body weight on the day of infection. The percentage of initial body weight curves were compared using a one-way analysis of variance (ANOVA) comparing each treatment group to placebo-treated hamsters. Tissue virus titers were compared using a two-way ANOVA comparing the treated animals to placebo-treated animals. Lung weights were compared using a two-way ANOVA.

Ethics regulation of laboratory animals: This study was conducted in accordance with the approval of the Institutional Animal Care and Use Committee of Utah State University dated Mar. 31, 2020 (expires Mar. 30, 2023). The work was done in the AAALAC-accredited Laboratory Animal Research Center of Utah State University. The U. S. Government (National Institutes of Health) approval was renewed Mar. 9, 2018 (PHS Assurance No. D16-00468[A3801-01]) in accordance to the National Institutes of Health Guide for the Care and Use of Laboratory Animals (Revision; 2011).

### Results and Discussion

This study evaluated treatment with berdazimer sodium on the reduction of lung virus titers, nasal tissue titers, and lung weights in hamsters infected with SARS-CoV-2. In addition, twice daily treatment with 2 mg/mL berdazimer sodium was compared to once daily treatment with 2 mg/mL berdazimer sodium.

In this study, a significant respiratory infection was observed as evidenced by high virus titers in the lung tissue and nasal tissue. Lung virus titers on day 3 post-infection were approximately three logs higher than the inoculum dose indicating a robust infection in the hamster respiratory tissue. In addition, weight loss was observed in the placebo-treated animals.

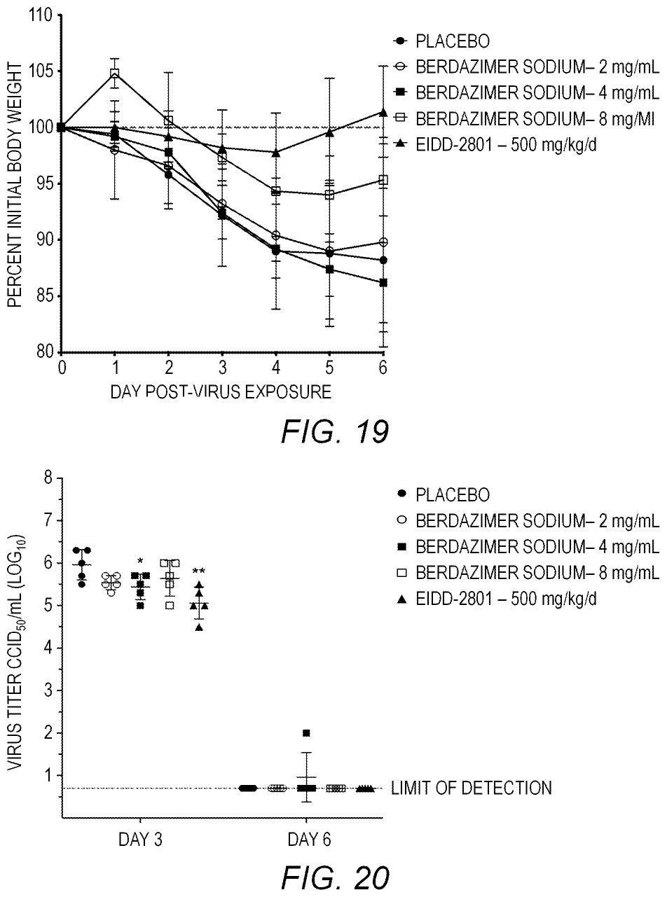

FIG. 19 shows percent initial body weight of hamsters treated once daily with berdazimer sodium (2, 4, or 8 mg/ml) and infected with SARS-CoV-2. Treatment with berdazimer sodium began 24 hours prior to infection. No statistically significant protection from weight loss was observed in hamsters treated with 2, 4, or 8 mg/mL of berdazimer sodium or with 500 mg/kg/d of EIDD-2801. Although not statistically significant, hamsters treated with 8 mg/mL of berdazimer sodium lost less body weight compared to placebo-treated animals. A similar trend was observed in the hamsters treated with EIDD-2801.

FIG. 20 shows lung virus titers of hamsters on day 3 and 6 treated once daily with berdazimer sodium (2, 4, or 8 mg/ml) and infected with SARS-CoV-2. Treatment with berdazimer sodium began 24 hours prior to infection. Treatment with berdazimer sodium at a dose of 4 mg/mL significantly reduced lung virus titers on day 3 post-infection. Treatment with EIDD-2801 at a dose of 500 mg/kg/d significantly reduced lung virus titers on day 3 post-infection. This one log₁₀ reduction in virus titers is comparable to other studies completed using EIDD-2801. Lung virus titers on Day 3 were lower in the animals that were treated once daily with 2 or 8 mg/mL of berdazimer sodium although the reductions were not statistically significant. Lung virus titers on Day 6 were below the limit of quantitation for all but one sample across all groups, suggesting clearance of the infection in this model by day 6.

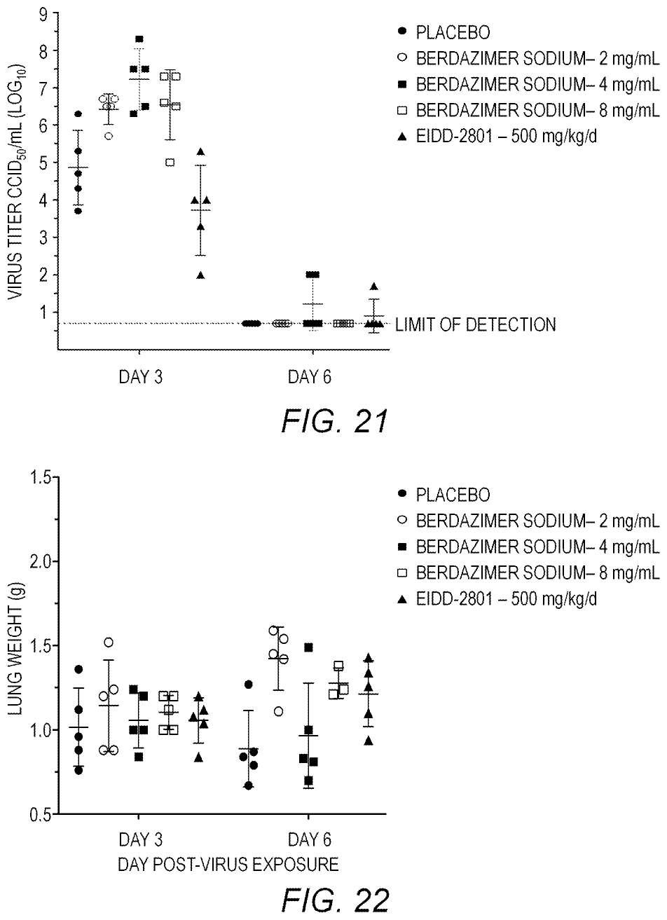

FIG. 21 shows nasal tissue virus titers of hamsters on day 3 and 6 treated with berdazimer sodium (2, 4, or 8 mg/ml) and infected with SARS-CoV-2. Treatment with berdazimer sodium began 24 hours prior to infection. Once daily treatment with berdazimer sodium at doses of 2, 4, or 8 mg/mL did not significantly reduce nasal tissue virus titers. Treatment with EIDD-2801 at a dose of 500 mg/kg/d did not significantly reduce nasal tissue virus titers.

FIG. 22 shows lung weights of hamsters on day 3 and 6 treated with berdazimer sodium (2, 4, or 8 mg/ml) and infected with SARS-CoV-2. Treatment with berdazimer sodium began 24 hours prior to infection. Once daily treatment with berdazimer sodium at doses of 2, 4, or 8 mg/mL did not significantly reduce lung weights. Treatment with EIDD-2801 at a dose of 500 mg/kg/d did not significantly reduce lung weights.

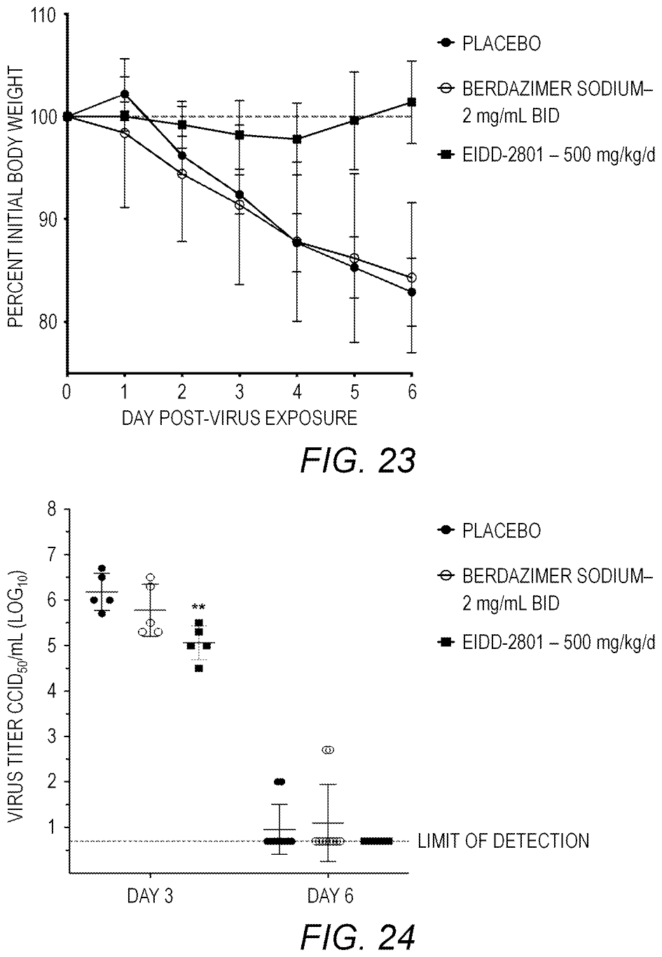

Percent initial body weight of hamsters treated twice daily with berdazimer sodium (2 mg/ml) and infected with SARS-CoV-2 are shown in FIG. 23. Treatment with berdazimer sodium began 24 hours prior to infection. Twice daily treatment with berdazimer sodium at a dose of 2 mg/mL did not prevent weight loss in hamsters infected with SARS-CoV-2. Although not statistically significant, hamsters treated with EIDD-2801 at a dose of 500 mg/kg/d lost less body weight than placebo-treated hamsters.

Lung virus titers of hamsters on day 3 and 6 treated twice daily with berdazimer sodium (2 mg/ml) and infected with SARS-CoV-2 are shown in FIG. 24. Treatment with berdazimer sodium began 24 hours prior to infection. Twice daily treatment with berdazimer sodium at a dose of 2 mg/mL did reduce lung virus titers on day 3 post-infection by approximately 12 log, however, the difference was not statistically significant. Treatment with EIDD-2801 at a dose of 500 mg/kg/d significantly reduced lung virus titers on day 3 post-infection.

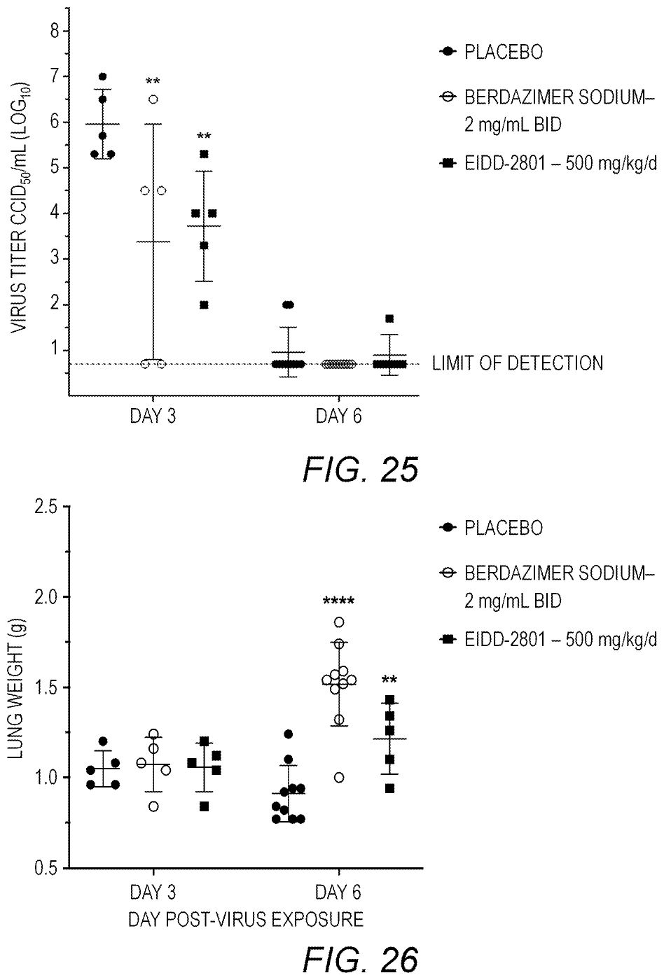

Nasal tissue virus titers of hamsters on day 3 and 6 treated twice daily with berdazimer sodium (2 mg/ml) and infected with SARS-CoV-2 are shown in FIG. 25. Treatment with berdazimer sodium began 24 hours prior to infection. Twice daily treatment with berdazimer sodium at a dose of 2 mg/mL significantly reduced nasal tissue virus titers on day 3 post-infection. Treatment with EIDD-2801 at a dose of 500 mg/kg/d significantly reduced nasal tissue virus titers on day 3 post-infection. There was an increased variability in the nasal tissue samples where some animals had high virus titers and some animals did not have detectable virus titers in nasal tissue. The small amount of tissue homogenized for these samples may have increased the variability. Table 12 shows the lung and nasal tissue virus titers on study days 3 and 6 of golden Syrian hamsters after treatment with berdazimer sodium and infection with SARS-CoV-2.

TABLE 12
Lung and nasal tissue virus titers on study days 3 and 6 of golden Syrian hamsters
after treatment with berdazimer sodium and infection with SARS-CoV-2.

| | | Virus Titer - *CCID₅₀/mL (Log₁₀) | | | | | | | | | | | | | | |
|---|---|---|---|---|---|---|---|---|---|---|---|---|---|---|---|---|
| | | Day 3 | | | | | Day 6 | | | | | | | | | |
| Treatment | Sample | 1 | 2 | 3 | 4 | 5 | 1 | 2 | 3 | 4 | 5 | 6 | 7 | 8 | 9 | 10 |
| Placebo - BID | Lung | 5.7 | 6.7 | 6.0 | 6.0 | 6.5 | 2.0 | 2.0 | <0.7 | <0.7 | <0.7 | <0.7 | <0.7 | <0.7 | <0.7 | <0.7 |
| Berdazimer sodium - 2 | tissue | 6.5 | 5.3 | 5.3 | 6.3 | 5.5 | <0.7 | <0.7 | <0.7 | 2.7 | <0.7 | <0.7 | <0.7 | 2.7 | <0.7 | <0.7 |
| mg/mL BID | | | | | | | | | | | | | | | | |
| Placebo - QD | | 6.0 | 6.3 | 5.5 | 5.7 | 6.3 | <0.7 | <0.7 | <0.7 | <0.7 | <0.7 | — | — | — | — | — |
| Berdazimer sodium - 2 | | 5.5 | 5.7 | 5.5 | 5.3 | 5.7 | <0.7 | <0.7 | <0.7 | <0.7 | <0.7 | — | — | — | — | — |
| mg/mL QD | | | | | | | | | | | | | | | | |
| Berdazimer sodium - 4 | | 5.7 | 5.5 | 5.0 | 5.7 | 5.3 | <0.7 | <0.7 | <0.7 | 2.0 | <0.7 | — | — | — | — | — |
| mg/mL QD | | | | | | | | | | | | | | | | |
| Berdazimer sodium - 8 | | 5.0 | 5.7 | 6.0 | 5.5 | 6.0 | <0.7 | <0.7 | <0.7 | X | X | — | — | — | — | — |
| mg/mL QD | | | | | | | | | | | | | | | | |
| EIDD-2801 - | | 5.0 | 4.5 | 5.3 | 5.0 | 5.5 | <0.7 | <0.7 | <0.7 | <0.7 | <0.7 | — | — | — | — | — |
| 500 mg/kg/d | | | | | | | | | | | | | | | | |
| Placebo - BID | Nasal | 5.3 | 7.0 | 6.5 | 5.7 | 5.3 | <0.7 | <0.7 | <0.7 | <0.7 | 2.0 | <0.7 | <0.7 | <0.7 | <0.7 | 2.0 |
| Berdazimer sodium - 2 | tissue | <0.7 | 4.5 | 4.5 | <0.7 | 6.5 | <0.7 | <0.7 | <0.7 | <0.7 | <0.7 | <0.7 | <0.7 | <0.7 | <0.7 | <0.7 |
| mg/mL BID | | | | | | | | | | | | | | | | |
| Placebo - QD | | 6.3 | 4.3 | 4.7 | 5.3 | 3.7 | <0.7 | <0.7 | <0.7 | <0.7 | <0.7 | — | — | — | — | — |
| Berdazimer sodium - 2 | | 6.5 | 6.7 | 6.5 | 6.7 | 5.7 | <0.7 | <0.7 | <0.7 | <0.7 | <0.7 | — | — | — | — | — |
| mg/mL QD | | | | | | | | | | | | | | | | |
| Berdazimer sodium - 4 | | 6.3 | 6.5 | 7.5 | 8.3 | 7.5 | <0.7 | 2.0 | <0.7 | 2.0 | <0.7 | — | — | — | — | — |
| mg/mL QD | | | | | | | | | | | | | | | | |
| Berdazimer sodium - 8 | | 7.3 | 6.6 | 7.3 | 6.5 | 5.0 | <0.7 | <0.7 | <0.7 | X | X | — | — | — | — | — |
| mg/mL QD | | | | | | | | | | | | | | | | |
| EIDD-2801 - 500 | | 3.3 | 4.0 | 4.0 | 2.0 | 5.3 | <0.7 | <0.7 | <0.7 | <0.7 | 1.7 | — | — | — | — | — |
| mg/kg/d | | | | | | | | | | | | | | | | |

*CCID₅₀—50% cell culture infectious dose
X—animals died prior to necropsy

Lung weights of hamsters on day 3 and 6 treated twice daily with berdazimer sodium (2 mg/ml) and infected with SARS-CoV-2 are shown in FIG. 26. Treatment with berdazimer sodium began 24 hours prior to infection. Lung weights were significantly increased on day 6 post-infection by twice daily treatment with berdazimer sodium. This is not likely due to the intranasal treatments since a similar increase in lung weights was observed in hamsters treated with EIDD-2801.

### Conclusions

This study evaluated the effect of once daily treatment with berdazimer sodium at 2, 4, and 8 mg/mL on weight loss, lung virus titers, nasal tissue titers, and lung weights. The effect of twice daily treatment with 2 mg/mL of berdazimer sodium was also evaluated.

Although not statistically significant, hamsters treated with 8 mg/mL of berdazimer sodium lost less weight than placebo-treated hamsters. However, two of the ten animals treated with 8 mg/mL of berdazimer sodium died on day 3 post-infection. Wild-type hamsters infected with SARS-CoV-2 do not normally succumb to infection but intranasal treatment may have exacerbated the infection. However, a prior study was conducted at the same regimen and dose level (8 mg/mL) and no premature deaths were observed.

A dose of 4 mg/mL of berdazimer sodium significantly reduced lung virus titers. Despite some variability between lung virus titers, a general trend appeared to show a dose-response to berdazimer sodium treatment. However, none of the doses of berdazimer sodium were able to reduce nasal tissue titers when administered once daily. When hamsters were treated twice daily with 2 mg/mL of berdazimer sodium, a significant reduction in nasal tissue titers was observed. Virus was not detected in the nasal tissue for two of the five animals that were infected with SARS-CoV-2 and treated with 2 mg/mL of berdazimer sodium twice daily.

### Example 5

The objectives of this study were to determine the potential toxicity of a composition (also referred to as test article) comprising berdazimer sodium (API) and a buffer, when the composition is administered intranasally 5 times a day for 14 days to dogs and to evaluate the potential reversibility of any findings. The buffer in the composition was a 200 mM citrate buffer having a pH of 4.5. The experimental design is provided in Table 13. The approximate daily dose levels were based on mean bodyweights of 8.5 kg for males and 6.5 kg for females, so the Daily Dose Level (mg/kg/day) was, for males, 0 mg/kg/day for Group 1, 0.47 mg/kg/day for Group 2, 0.94 mg/kg/day for Group 3, and 1.65 mg/kg/day for Group 4, and, for females, 0 mg/kg/day for Group 1, 0.61 mg/kg/day for Group 2, 1.23 mg/kg/day for Group 3, and 2.15 mg/kg/day for Group 4.

TABLE 13
Experimental Design.

| | | | Total | | | | | | |
|---|---|---|---|---|---|---|---|---|---|
| | | Dose | Dose | | | No. of Animals | | | |
| | | Level^{a} | Level | Dose | Dose | | | Recovery | |
| Group | Test | (API | (API | Volume^{b} | Concentration | Main Study | | Study | |
| No. | Material | mg/dose) | mg/day) | (mL/dose) | (API mg/mL) | Males | Females | Males | Females |
| 1 | Control | 0 | 0 | 0.2 | 0 | 3 | 3 | 2 | 2 |
| | Article^{c} | | | | | | | | |
| 2 | Test | 0.8 | 4 | 0.2 | 4 | 3 | 3 | — | — |
| | article | | | | | | | | |
| 3 | Test | 1.6 | 8 | 0.2 | 8 | 3 | 3 | — | — |
| | article | | | | | | | | |
| 4 | Test | 2.8 | 14 | 0.2 | 14 | 3 | 3 | 2 | 2 |
| | article | | | | | | | | |

^{a}Dosed intranasally 5 times a day (2 hours ± 30 minutes apart) for 14 days.
^{b}Dose volume was equally split between both nostrils.
^{c}200 mM Citrate Buffer, pH 4.5.

The following parameters and end points were evaluated in this study: mortality, clinical observations, body weights, body weight gains, food consumption, ophthalmology, electrocardiology, clinical pathology parameters (hematology, coagulation, clinical chemistry, methemoglobin, and urinalysis), toxicokinetic parameters, organ weights, and macroscopic and microscopic examinations.

In regard to the test articles, there were no changes for mortality, clinical observations, body weights, body weight gains, food consumption, ophthalmology, clinical pathology (hematology, coagulation, clinical chemistry, and urine), organ weights, and macroscopic examinations, and there were no adverse findings for the microscopic examinations.

For ECGs, there was a statistically significant longer QRS complex duration noted for Group 4 (14 mg/day) males at 1 to 2 hours postdose on Day 11. This change was considered to be test material related based on the magnitude of change, but was considered nonadverse since it resolved during the recovery period.

On Day 14, all MetHgb values for the 14 mg/day male and female dogs decreased, as compared to baseline values, at each postdose collection interval, with the exception of a slight increase for the female dogs at the 2-hour collection interval, just prior to dosing. Additionally, all control male and female dogs exhibited a decrease in MetHgb, as compared to baseline values, at each postdose collection interval on Day 14.

At 14 mg/day, there were test article-related microscopic changes in the nasal turbinates of the nasal cavity, including mild to moderate mixed inflammation and minimal to mild luminal exudate in male and female dogs that were considered non-adverse. These findings exhibited complete recovery in the male dogs but were without complete recovery in the female dogs at the end of the 7-day recovery period, although exudate appeared to be resolving in the females during the recovery period.

In conclusion, administration of the test article 5 times daily by intranasal administration for 14 days was well tolerated in dogs at levels of 14 mg of API/day. Target organ effects were observed at levels of 14 mg/day and consisted of test article-related microscopic changes in the nasal turbinates of the nasal cavity, including mild to moderate mixed inflammation and minimal to mild luminal exudate in male and female dogs that were considered non-adverse. These findings exhibited complete recovery in the male dogs but only partial recovery in the female dogs at the end of the 7-day recovery period. Additionally, a statistically significant longer QRS complex duration was noted for 14 mg/day male dogs at 1 to 2 hours postdose on Day 11. This change was considered to be test material related based on the magnitude of change, but the change resolved during the recovery period. Based on these results, the no-observed-adverse-effect level (NOAEL) was considered to be 14 mg/day (i.e., 1.65 mg/kg/day in males and 2.15 mg/kg/day in females). At the NOAEL, the mean Cₘₐₓ and AUC₍₀₋₂₄ₕ₎ values for males were 11,800 pg/mL and 179,000 h·pg/mL, and for females 10,700 pg/mL and 173,000 h·pg/mL, respectively, after 14 days of treatment.

### Example 6

Buffer concentration, pH, and osmolality were evaluated in view of the alkaline API (berdazimer sodium). For this study, test formulations 11b-17b were prepared at multiple berdazimer sodium concentrations between 2 mg/mL and 18 mg/mL, using only buffer as the test formulation vehicle. All samples were prepared w/v by weighing API into glass vials and diluting with 10 mL of the respective buffer to target the necessary berdazimer sodium concentration. The samples were then assessed for test formulation pH and osmolality. The vehicle samples were also assessed for pH and osmolality. For test formulations 6b and 7b, the samples were prepared at a 2 mg/mL and 24 mg/mL concentration. All samples were prepared w/v by weighing API into glass vials and diluting with 5 mL of the respective buffer to target the necessary berdazimer sodium concentration. The samples were then assessed for test formulation pH and osmolality. The vehicle samples were also tested for pH and osmolality.

The test formulations evaluated can be seen in Table 14.

### 2 mg mL Samples

Upon compounding, foaming was not observed, and all samples produced a finely dispersed suspension. In terms of pH, the pH of all samples returned below the target pH of pH 5.50, with all samples returning around pH 4.60-4.80. In terms of osmolality, formulation osmolality values increased with increasing buffer concentration. All osmolality values, however, returned within the tolerable range for intranasal dosing.

### 8 mg mL Samples

Upon compounding, minimal foaming comprising of a thin consistency was observed in all samples. In terms of API wetting and dispersibility, the API readily wetted, creating a finely dispersed suspension. In terms of pH, all formulations except 11b, returned near the target pH of pH 5.50. The osmolality trends returned similar to the 2 mg/mL samples, with values increasing with increasing buffer concentration. All osmolality values returned within the tolerable range for intranasal dosing.

### 12 mg mL Samples

Upon compounding, a large amount of thick foam was produced in test formulations 15b and 16b, while a small amount of foam was produced in test formulations 17b and 1 lb. In terms of API wetting and dispersibility, the API readily wetted, creating a finely dispersed suspension. In terms of pH, all test formulations expect 15b returned near the target pH of pH 5.50. The pH value of test formulation 15b returned at pH 7.07, suggesting that a 100 mM buffer concentration is not strong enough to buffer against a 12 mg/mL API concentration. In terms of osmolality, similar trends were observed, and all values returned within the tolerable range for intranasal dosing.

### 14 mg mL Samples

Upon compounding, a large amount of thick foam was produced in test formulations 15b-17b, while a small amount of foam being produced in test formulation 1 lb. In terms of API wetting and dispersibility, the API readily wetted, creating a finely dispersed suspension. In terms of pH, all test formulations expect 15b returned near the target pH of pH 5.50. In terms of osmolality, similar trends were observed, and all values returned within the tolerable range for intranasal dosing.

### 16 mg mL Samples

Upon compounding, a large amount of thick foam was produced in test formulations 15b and 1 Fb, while a small amount of foam was produced in test formulations 16b and 17b. In terms of API wetting and suspensibility, a large amount of dry API remained at the bottom of each sample and could not be mixed and suspended due to the thick layer of foam residing at the top of each vial. In terms of pH, all test formulations expect 15b returned near the target pH of pH 5.50. In terms of osmolality, similar trends were observed, and all values returned within the tolerable range for intranasal dosing.

18 mg/ML Samples

Upon compounding, a large amount of thick foam was produced in all samples. In terms of API wetting and suspensibility, a large amount of dry API remained at the bottom of each sample and could not be mixed and suspended due to the thick layer of foam residing at the top of each vial. In terms of pH, all test formulations returned near the target pH of pH 5.50. In terms of osmolality, similar trends were observed, and all values returned within the tolerable range for intranasal dosing.

All data generated from this study can be seen in Tables 15-22.

TABLE 14
Test Formulation Compositions

| | | Berdazimer |
|---|---|---|
| | | Sodium |
| Form | | Concentration |
| No. | Buffer Concentration | (mg/mL) |
| 6b | 0.15% Sodium | 2 |
| | carboxymethyl cellulose (Na | |
| | CMC) + 1% Hydroxypropyl | |
| | cellulose (HPC) in 50 mM | 24 |
| | Citrate Buffer, pH 4.50 | |
| 7b | 0.15% Na CMC + 1% HPC | 2 |
| | in 100 mM Citrate Buffer, | |
| | pH 5.50 | 24 |
| 15b | 100 mM Citrate Buffer, | 2 |
| | pH 4.50 | 8 |
| | | 12 |
| | | 14 |
| | | 16 |
| 16b | 200 mM Citrate Buffer, | 2 |
| | pH 4.50 | 8 |
| | | 12 |
| | | 14 |
| | | 16 |
| 17b | 250 mM Citrate Buffer, | 2 |
| | pH 4.50 | 8 |
| | | 12 |
| | | 14 |
| | | 16 |
| | | 18 |
| 11b | 300 mM Citrate Buffer, | 8 |
| | pH 4.50 | 12 |
| | | 14 |
| | | 16 |
| | | 18 |

TABLE 15
2 mg/mL pH and Osmolality Values

| | API | | Formulation | |
|---|---|---|---|---|
| Form | Concentration | pH After | Osmolality | |
| # | (mg/mL) | Compounding | (mmol/kg) | Appearance Observations |
| 15b | 2 | 4.81 | 255 | Finely dispersed suspension, |
| | | | | no foaming |
| 16b | | 4.64 | 475 | Finely dispersed suspension, |
| | | | | no foaming |
| 17b | | 4.65 | 578 | Finely dispersed suspension, |
| | | | | no foaming |

TABLE 16
8 mg/mL pH and Osmolality Values

| | API | | Formulation | |
|---|---|---|---|---|
| Form | Concentration | pH After | Osmolality | |
| # | (mg/mL) | Compounding | (mmol/kg) | Appearance Observations |
| 15b | 8 | 5.82 | 302 | API easily wetted, thin |
| | | | | consistency foam was produced. |
| | | | | Majority of foam dissipated |
| | | | | after~5 minutes |
| 16b | | 5.12 | 504 | API easily wetted, thin |
| | | | | consistency foam was produced. |
| | | | | Majority of foam dissipated |
| | | | | after~5 minutes |
| 17b | | 5.01 | 607 | API easily wetted, thin |
| | | | | consistency foam was produced. |
| | | | | Majority of foam dissipated |
| | | | | after~5 minutes |
| 11b | | 4.87 | 730 | API easily wetted, thin |
| | | | | consistency foam was produced |

TABLE 17
12 mg/mL pH and Osmolality Values

| | API | | Formulation | |
|---|---|---|---|---|
| Form | Concentration | pH After | Osmolality | |
| # | (mg/mL) | Compounding | (mmol/kg) | Appearance Observations |
| 15b | 12 | 7.07 | 309 | API easily wetted, large amount |
| | | | | of thick foaming was produced |
| 16b | | 5.42 | 526 | API easily wetted, large amount |
| | | | | of thick foaming was produced |
| 17b | | 5.18 | 645 | API easily wetted, small |
| | | | | amount of thick foaming was |
| | | | | produced |
| 11b | | 5.10 | 743 | API easily wetted, small |
| | | | | amount of thick foaming was |
| | | | | produced |

TABLE 18
14 mg/mL pH and Osmolality Values

| | API | | Formulation | |
|---|---|---|---|---|
| Form | Concentration | pH After | Osmolality | |
| # | (mg/mL) | Compounding | (mmol/kg) | Appearance Observations |
| 15b | 14 | 7.78 | 329 | API easily wetted, large amount |
| | | | | of thick foaming was produced |
| 16b | | 5.55 | 543 | API easily wetted, large amount |
| | | | | of thick foaming was produced |
| 17b | | 5.40 | 650 | API easily wetted, large amount |
| | | | | of thick foaming was produced |
| 11b | | 5.21 | 757 | API easily wetted, small |
| | | | | amount of thick foaming was |
| | | | | produced |

TABLE 19
16 mg/mL pH and Osmolality Values

| | API | | Formulation | |
|---|---|---|---|---|
| Form | Concentration | pH After | Osmolality | |
| # | (mg/mL) | Compounding | (mmol/kg) | Appearance Observations |
| 15b | 16 | 8.59 | 341 | API did not fully wet and |
| | | | | suspend, large amount of thick |
| | | | | foaming was produced |
| 16b | | 5.78 | 545 | API did not fully wet and |
| | | | | suspend, small amount of thick |
| | | | | foaming was produced |
| 17b | | 5.50 | 663 | API did not fully wet, small |
| | | | | amount of thick foaming was |
| | | | | produced |
| 11b | | 5.25 | 768 | API did not fully wet, large |
| | | | | amount of thick foaming was |
| | | | | produced |

TABLE 20
18 mg/mL pH and Osmolality Values

| | API | | Formulation | |
|---|---|---|---|---|
| Form | Concentration | pH After | Osmolality | |
| # | (mg/mL) | Compounding | (mmol/kg) | Appearance Observations |
| 17b | 18 | 5.74 | 654 | API did not fully wet and |
| | | | | suspend, thick foaming was |
| | | | | produced |
| 11b | | 5.46 | 764 | API did not fully wet and |
| | | | | suspend, thick foaming was |
| | | | | produced |

TABLE 21
Test Formulation Vehicle Osmolality Values

| | API | Vehicle | Osmolality |
|---|---|---|---|
| Sample Type | Concentration | pH | (mmol/kg) |
| Vehicle 15b | N/A | 4.51 | 225 |
| Vehicle 16b | | 4.55 | 448 |
| Vehicle 17b | | 4.48 | 554 |
| Vehicle 11b | | 4.50 | 660 |

TABLE 22
Vehicle and Test Formulation 6b and 7b Osmolality Values

| | API | | |
|---|---|---|---|
| Form | Concentration | pH After | Osmolality |
| # | (mg/mL) | Compounding | (mmol/kg) |
| 6b | N/A | 4.55 | 123 |
| 7b | | 5.50 | 269 |
| 6b | 2 | 4.96 | 154 |
| 7b | | 5.72 | 309 |
| 6b | 24 | 10.49 | 429 |
| 7b | | 9.45 | 611 |

### Summary and Conclusions:

The results of this study indicate that the upper API limit for excessive foaming lies between 8 mg/mL and 12 mg/mL for the tested buffers. Based off the pH data, a 200 mM citrate buffer appears to effectively buffer against the API at the 2 mg/mL to 16 mg/mL concentration range and maintain a formulation pH near the target pH of pH 5.50. In terms of API wetting and dispersibility, API concentrations above 14 mg/mL do not allow for adequate API suspensibility, resulting in large amounts of dry API remaining at the bottom of the vials. A 14 mg/mL API concentration allows for full API wetting and suspensibility, while also maintaining the target test formulation pH.

### Example 7

The antiviral activity of berdazimer sodium was evaluated against Respiratory syncytial virus (RSV-A2) and Influenza A/California/7/2009 (H1N1) in a highly differentiated, three-dimensional (3-D), in vitro model of normal, human-derived tracheal/bronchial epithelial (TBE) cells. The compound was tested at various concentrations in triplicate inserts of the 3D tissue models of human EpiAirway (MatTek Life Sciences). Antiviral activity was measured by virus yield reduction assays 3 (H1N1) and 6 (RSV-A2) days after infection.

### Materials and Methods

Compounds: The compound (berdazimer sodium) was received as a solid and stored at −20° C. until prepared. For each day, fresh berdazimer sodium drug was prepared by dissolving the compound in 100% dimethyl sulfoxide (DMSO) and then further diluting to the test dilutions in the MatTek culture medium (AIR-100-MM).

Cell Culture: The EpiAirway™ Model consists of normal, human-derived tracheal/bronchial epithelial (TBE) cells which have been cultured to form a multi layered, highly differentiated model which closely resembles the epithelial tissue of the respiratory tract. The cell cultures were made to order by MatTek Life Sciences (https://www.mattek.com) (Ashland, MA) and arrived in kits with either 12- or 24-well inserts each. The TBE cells were grown on 6 mm mesh disks in transwell inserts. During transportation, the tissues were stabilized on a sheet of agarose, which was removed upon receipt, 24 hours after being shipped. One insert was estimated to consist of approximately 1.2×10⁶ cells. Kits of cell inserts (EpiAirway™ AIR-100, AIR-112) originated from a single, healthy, non-smoker donor #9831.

Upon arrival, the cell transwell inserts were immediately transferred to individual wells of a 6-well plate according to manufacturer's instructions. 1 mL of MatTek's proprietary culture medium (AIR-100-MM) was then added to the basolateral side, whereas the apical side was exposed to a humidified 5% CO₂ environment. The TBE cells were cultured at 37° C. for two days before the start of the experiment. After the 48-hour equilibration period, the mucin layer, secreted from the apical side of the cells, was removed by washing three times with 400 μL pre-warmed 30 mM HEPES buffered saline solution. Culture medium was replenished to the basal side following the wash steps. The tissues were then allowed to rest in a 37° C. and 5% CO₂ environment for a minimum of 1 hour prior to the assay.

Viruses: RSV-A2 (ATCC VR-1540) was passaged twice in MA-105 cells to create the virus stock. The virus dose that was able to infect 50% of the cell cultures (CCID₅₀ per 0.2 mL) was calculated by the Reed-Muench method (1938). The virus stock was then diluted in AIR-100-MM and infected at MOI 0.1 CCID₅₀ per cell. Influenza A (H1N1) was passaged twice in MDCK cells to create the virus stock. The virus dose that was able to infect 50% of the cell cultures (CCID₅₀ per 0.2 mL) was calculated by the Reed-Muench method (1938). The virus stock was then diluted in AIR-100-MM and infected at MOI 0.001 CCID₅₀ per cell.

Experimental design: Each compound treatment (140 μL) and virus (140 μL) was applied to the apical side, and compound treatment only was applied to the basal side (1 mL), for a 2-hour infection period. As virus controls, 3 of the cell wells were treated with placebo (cell culture medium only) for each virus. Following the 2-hour infection, the apical medium was removed, and the basal side was replaced with freshly prepared compound or medium. The cells were maintained at the air-liquid interface. Each day at approximately the same time (within 1-2 hours) the basal side was removed and replaced with freshly prepared compound or fresh media (virus controls). [Note that the data shown in Table 23 is the result after the basal side was not replaced with fresh compound on days 3 and 4.] On day 3 (H1N1) or day 6 (RSV), the medium was removed and discarded from the basal side. Virus released into the apical compartment of the tissues was harvested by the addition of 400 μL of culture medium that was pre-warmed at 37° C. The contents were incubated for 30 minutes, mixed well, collected, thoroughly vortexed, and frozen at −80° C. for eventual plating on MDCK cells (H1N1) or on MA-105 cells (RSV) the same day as collected for virus yield reduction (VYR) titration. Ribavirin was included in this study as a positive control for antiviral effect.

Determination of virus titers from each treated cell culture: Cells were seeded in 96-well plates and grown overnight (37° C.) to 90% confluence. Samples containing virus were diluted in 10-fold increments in infection medium and 200 μL of each dilution was transferred into respective wells of a 96-well microtiter plate. Four microwells were used for each dilution to determine 50% viral endpoints. After 3 days (H1N1) or 7 days (RSV) of incubation, each well was scored positive for virus if any cytopathic effect (CPE) was observed as compared with the uninfected control. The 90% effective concentration (EC₉₀), or amount of compound required to reduce virus yield by 1 log₁₀, was determined by regression analysis.

Cytotoxicity assay: The CCK-8 colorimetric assay is based on the reduction of highly water-soluble tetrazolium salt, WST-8, which produces orange water-soluble formazan dye in the presence of an electron mediator. The amount of formazan is directly proportional to the number of living cells and the resulting colored solution is quantified by measuring absorbance at 450 nanometers using a multi-well spectrophotometer. Drug concentrations of 2 mg/ml and 1 mg/ml were tested in duplicate wells, and a drug concentration of 0.5 mg/ml was tested in singlet wells. On day 3 (H1N1) or day 6 (RSV), the apical side was washed one time with 400 μL pre-warmed PBS. The CCK-8 solution was added (200 L) to the apical side and incubated at 37° C. for 2 hours. The formazan (100 l) was placed into a 96-well plate and the 450 nm absorbance was recorded. Percent toxicity values were calculated as a percentage of the cell control test wells (no drug treatment) and reported as the 50% cell cytotoxic concentration (CC₅₀) of compound (no virus). Tissues were also observed via microscopy to assess cytotoxicity.

The VYR titer data, EC₉₀ and CC₅₀ values are summarized in Tables 23-25.

### Conclusions

The EC₉₀ values from berdazimer sodium treatment on days 1, 2, and 5 of RSV-A2 infection were 0.79-1.07 mg/mL, as shown in Table 23, while the EC₉₀ values from berdazimer sodium treatment on days 1 through 5 of RSV-A2 infection were 0.96-1.44 mg/mL, as shown in Table 24. While not wishing to be bound to any particular theory, no difference was observed in efficacy for the two RSV-A2 treatment regimens under the conditions tested. As shown in Table 25, the EC₉₀ values from berdazimer sodium treatment on days 1 and 2 of H1N1 infection were 1.11-1.14 mg/mL. Tissues observed under the microscope showed no overt cytotoxicity.

TABLE 23
Antiviral efficacy against Respiratory syncytial virus strain A2 (RSV-A2)
when fresh drug was prepared on days 1, 2, and 5 of the infection.

| | | ^{a}Log₁₀ | | | |
|---|---|---|---|---|---|
| | | CCID₅₀ | | | |
| | | virus | | | |
| Test | Concentration | per 0.2 | ^{b}EC₉₀ | ^{c}CC₅₀ | |
| Compound | (mg/ml) | mL | (mg/ml) | mg/ml) | ^{d}SI |
| Berdazimer | 2 | 2.67 | 0.79 | 2.0 | 2.54 |
| Sodium | 1 | 3.00 | | | |
| | 0.5 | 3.67 | | | |
| | 0.1 | 4.67 | | | |
| Berdazimer | 2 | 2.30 | 1.0 | 2.0 | 2.00 |
| Sodium | 1 | 3.00 | | | |
| | 0.5 | 4.50 | | | |
| | 0.1 | 4.30 | | | |
| Berdazimer | 2 | 3.50 | 1.07 | 2.0 | 1.87 |
| Sodium | 1 | 3.30 | | | |
| | 0.5 | 3.67 | | | |
| | 0.1 | 4.50 | | | |
| Ribavirin | 100 (μg/ml) | 1.67 | 5.12 μg/ml | >100 μg/ml | >19.54 |
| | 10 (μg/ml) | 2.67 | | | |
| | 1 (μg/ml) | 4.30 | | | |
| Virus | MOI 0.1 | 4.30 | Avg. | | |
| Control | | 4.00 | 4.27 | | |
| RSV-A2 | | 4.50 | | | |

Each well was scored positive for virus if any CPE was observed as compared with the uninfected control.
^{a}Titer results from the virus yield reduction (VYR) assay.
^{b}EC₉₀ = VYR 90% effective concentration (reduce virus yield by 1 log₁₀) as determined by regression analysis.
^{c}CC₅₀ = 50% cell cytotoxic concentration of compound (no virus)
^{d}SI = CC₅₀/EC₉₀; Selectivity Index is a measure of the window between cytotoxicity and antiviral activity. The higher the SI ratio, the more effective and safe a drug would be during in vivo treatment for a given viral infection.

TABLE 24
Antiviral efficacy against Respiratory syncytial virus strain A2
(RSV-A2) when fresh drug was prepared on days 1-5 of the infection.

| | | ^{a}Log₁₀ | | | |
|---|---|---|---|---|---|
| | | CCID₅₀ | | | |
| | | virus | | | |
| Test | Concentration | per 0.2 | ^{b}EC₉₀ | ^{c}CC₅₀ | |
| Compound | (mg/ml) | mL | (mg/ml) | mg/ml) | ^{d}SI |
| Berdazimer | 2 | 3.30 | 0.96 | 1.9 | 1.97 |
| Sodium | 1 | 3.50 | | | |
| | 0.5 | 4.00 | | | |
| | 0.1 | 4.67 | | | |
| Berdazimer | 2 | 3.30 | 1.44 | 1.9 | 1.32 |
| Sodium | 1 | 3.67 | | | |
| | 0.5 | 4.30 | | | |
| | 0.1 | 4.30 | | | |
| Berdazimer | 2 | 3.50 | 1.01 | 1.9 | 1.88 |
| Sodium | 1 | 3.50 | | | |
| | 0.5 | 4.30 | | | |
| | 0.1 | 4.67 | | | |
| Ribavirin | 100 (μg/ml) | 2.00 | 4.6 μg/ml | >100 μg/ml | >21.7 |
| | 10 (μg/ml) | 3.00 | | | |
| | 1 (μg/ml) | 4.30 | | | |
| Virus | MOI 0.1 | 4.30 | Avg. | | |
| Control | | 4.50 | 4.49 | | |
| RSV-A2 | | 4.67 | | | |

Each well was scored positive for virus if any CPE was observed as compared with the uninfected control.
^{a}Titer results from the virus yield reduction (VYR) assay.
^{b}EC₉₀ = VYR 90% effective concentration (reduce virus yield by 1 log₁₀) as determined by regression analysis.
^{c}CC₅₀ = 50% cell cytotoxic concentration of compound (no virus)
^{d}SI = CC₅₀/EC₉₀; Selectivity Index is a measure of the window between cytotoxicity and antiviral activity. The higher the SI ratio, the more effective and safe a drug would be during in vivo treatment for a given viral infection.

TABLE 25
Antiviral efficacy against Influenza A/California/7/2009 (H1N1)
when fresh drug was prepared on days 1 and 2 of the infection.

| | | ^{a}Log₁₀ | | | |
|---|---|---|---|---|---|
| | | CCID₅₀ | | | |
| | | virus | | | |
| Test | Concentration | per 0.2 | ^{b}EC₉₀ | ^{c}CC₅₀ | |
| Compound | (mg/ml) | mL | (mg/ml) | mg/ml) | ^{d}SI |
| Berdazimer | 2 | 3.00 | 1.14 | >2.0 | >1.75 |
| Sodium | 1 | 6.50 | | | |
| | 0.5 | 7.00 | | | |
| | 0.1 | 7.00 | | | |
| Berdazimer | 2 | 2.00 | 1.13 | >2.0 | >1.76 |
| Sodium | 1 | 6.67 | | | |
| | 0.5 | 7.00 | | | |
| | 0.1 | 6.67 | | | |
| Berdazimer | 2 | 2.00 | 1.11 | >2.0 | >1.80 |
| Sodium | 1 | 6.50 | | | |
| | 0.5 | 6.67 | | | |
| | 0.1 | 7.00 | | | |
| Ribavirin | 100 (μg/ml) | 2.67 | 1.89 μg/ml | >100 μg/ml | >52.9 |
| | 10 (μg/ml) | 4.00 | | | |
| | 1 (μg/ml) | 6.50 | | | |
| Virus | MOI 0.001 | 6.50 | Avg. | | |
| Control | | 7.30 | 6.82 | | |
| Influenza A | | 6.67 | | | |
| H1N1 | | | | | |

Each well was scored positive for virus if any CPE was observed as compared with the uninfected control.
ªTiter results from the virus yield reduction (VYR) assay.
^{b}EC₉₀ = VYR 90% effective concentration (reduce virus yield by 1 log10) as determined by regression analysis.
^{c}CC₅₀ = 50% cell cytotoxic concentration of compound (no virus)
^{d}SI = CC₅₀/EC₉₀; Selectivity Index is a measure of the window between cytotoxicity and antiviral activity. The higher the SI ratio, the more effective and safe a drug would be during in vivo treatment for a given viral infection.

The foregoing is illustrative of the present invention, and is not to be construed as limiting thereof. The invention is defined by the following claims, with equivalents of the claims to be included therein. All publications, patent applications, patents, patent publications, and other references cited herein are incorporated by reference in their entireties for the teachings relevant to the sentence and/or paragraph in which the reference is presented.

That which is claimed is:

1. A composition comprising:
a nitric oxide-releasing active pharmaceutical ingredient; and
a buffer configured to maintain the pH of the composition in a range of about 3, 3.5, 4, or 4.5 to about 5, 5.5., 6, 6.5, 7, 7.5, 8, or 8.5.

2. The composition of claim 1, wherein the buffer has a pH of about 4 to about 6 and/or the buffer comprises a weak acid that has at least two pKa values in a range of about 2.5 to about 6.5.

3. A composition comprising:
a nitric oxide-releasing active pharmaceutical ingredient; and
a buffer, the buffer comprising a citrate (e.g., a citrate salt) and/or citric acid in an amount of at least 100 mM.

4. The composition of claim 3, wherein the buffer has a pH of about 4 to about 6, optionally wherein the buffer has a pH of about 4.5.

5. The composition of claim 3 or 4, wherein the buffer comprises the citrate and/or citric acid in an amount of at least about 100 mM to about 300 mM, optionally wherein the buffer comprises the citrate and/or citric acid in an amount of about 200 mM.

6. The composition of any one of claims 3-5, wherein the citrate and/or citric acid is selected from citric acid, trisodium citrate, potassium citrate, calcium citrate, and/or a hydrate thereof (e.g., tri sodium citrate dihydrate) or an anhydrous form thereof, optionally wherein the citrate and/or citric acid is sodium citrate tribasic (e.g., anhydrous sodium citrate tribasic) and citric acid.

7. The composition of any one of claims 3-6, wherein the buffer comprises citric acid in an amount of about 1%, 1.25%, 1.5%, or 1.75% to about 2%, 2.25%, or 2.5% w/w; sodium citrate tribasic and/or tri sodium citrate dihydrate in an amount of about 1.5%, 1.75%, 2%, 20.25%, 2.5%, 20.75%, or 3% to about 30.25%, 3.5%, 30.75%, or 4% w/w; optionally an additional acid (e.g., HCl) and/or base (e.g., NaOH) in an amount sufficient to adjust the pH of the buffer to about 4 to about 6 (e.g., about 4.5); and a remainder of water.

8. The composition of any one of claims 3-7, wherein the buffer consists of water, citric acid, optionally citrate, optionally an additional acid (e.g., HCl), and optionally a base (e.g., NaOH).

9. The composition of any preceding claim, wherein the nitric oxide-releasing active pharmaceutical ingredient is suspended in the buffer.

10. The composition of any preceding claim, wherein the nitric oxide-releasing active pharmaceutical ingredient is present in the composition in an amount of about 0.1 mg/mL to about 30 mg/mL, optionally about 1 mg/mL to about 20 mg/mL.

11. The composition of any preceding claim, wherein the nitric oxide-releasing active pharmaceutical ingredient has a particle size (e.g., diameter) of about 20 nm to about 30 μm, optionally a mean particle size (e.g., mean diameter) of about 2 μm to about 20 μm.

12. The composition of any preceding claim, wherein the composition has an initial pH of about 4.5 to about 6.

13. The composition of any preceding claim, wherein the composition has a pH of about 4.5 to about 8.5, optionally wherein the composition has a pH of about 5.5.

14. The composition of any preceding claim, wherein the composition is configured to be aerosolized and/or atomized, optionally wherein the composition is configured for intranasal administration.

15. The composition of any preceding claim, wherein the composition is antiviral, optionally wherein the buffer is not antiviral and/or viricidal.

16. The composition of any preceding claim, wherein the composition consists of the nitric oxide-releasing active pharmaceutical ingredient and the buffer.

17. The composition of any preceding claim, wherein the buffer and/or composition are devoid of a diluent (e.g., devoid of a diluent other than the buffer), a co-solvent (e.g., devoid of a solvent other than the buffer), a preservative, an antioxidant, a suspending agent, a penetration enhancer, a surfactant, a viscosity-increasing agent, a humectant, a stabilizer, and/or a wetting agent.

18. The composition of any preceding claim, wherein the composition releases nitric oxide in an amount of about 0.001% to about 10% by weight of the composition, as measured by real time in vitro release testing, and/or the nitric oxide-releasing active pharmaceutical ingredient stores and/or releases nitric oxide in an amount of about 0.001% to about 10% by weight of the nitric oxide-releasing active pharmaceutical ingredient and/or composition, as measured by real time in vitro release testing.

19. The composition of any preceding claim, wherein the nitric oxide-releasing active pharmaceutical ingredient comprises a diazeniumdiolate functionalized co-condensed silica network.

20. The composition of any preceding claim, wherein the nitric oxide-releasing active pharmaceutical ingredient comprises a co-condensed silica network comprising diazeniumdiolated methylaminopropyl trimethoxysilane (MAP3-NONOate) and tetraethyl orthosilicate (TEOS).

21. The composition of any preceding claim, wherein the nitric oxide-releasing active pharmaceutical ingredient comprises a co-condensed silica network comprising diazeniumdiolated methylaminopropyl trimethoxysilane (MAP3-NONOate), ethylaminoisobutylsiloxane (EAIB3), and tetraethyl orthosilicate (TEOS).

22. The composition of any preceding claim, wherein the composition administers nitric oxide in an amount sufficient to induce apoptosis in virally infected cells.

23. The composition of any preceding claim, wherein the composition administers nitric oxide in an amount sufficient to reduce or eliminate viral replication, optionally with less than about 50% host cell cytotoxicity.

24. A kit comprising:
a first composition comprising a nitric oxide-releasing active pharmaceutical ingredient; and
a second composition comprising a buffer configured to maintain the pH of the composition in a range of about 3, 3.5, 4, or 4.5 to about 5, 5.5., 6, 6.5, 7, 7.5, 8, 8.5,
wherein the first composition and second composition are separately stored in the kit.

25. A kit comprising:
a first composition comprising a nitric oxide-releasing active pharmaceutical ingredient; and
a second composition comprising a buffer comprising a citrate and/or citric acid in an amount of at least 100 mM,
wherein the first composition and second composition are separately stored in the kit.

26. The kit of claim 24 or 25, wherein the first composition is a solid or in a particulate form.

27. The kit of any one of claims 24-26, wherein the second composition is a solution.

28. The kit of any one of claims 24-27, wherein the buffer has a pH of about 4 to about 6, optionally wherein the buffer has a pH of about 4.5, and/or wherein the buffer comprises a weak acid having at least two pKa values in a range of about 2.5 to about 6.5.

29. The kit of any one of claims 24-28, wherein the kit is configured to administer and/or release a volume of about 15 μL to about 150, 200, 300, 400, or 500 μL and/or the kit comprises a device configured to administer and/or release a volume of about 15 μL to about 150, 200, 300, 400, or 500 μL, optionally wherein the kit and/or device is configured to combine the first and second compositions.

30. The kit of any one of claims 24-29, wherein the kit is configured to aerosolize and/or atomize a composition comprising the first and second compositions and/or the kit comprises a device configured to aerosolize and/or atomize a composition comprising the first and second compositions, optionally wherein the aerosol and/or atomized composition has a mean droplet size (e.g., droplet diameter) of about 10, 20, 30, or 40 μm to about 100 μm.

31. The kit of any one of claims 24-30, wherein the buffer has a pH of about 4 to about 6, optionally wherein the buffer has a pH of about 4.5.

32. The kit of any one of claims 25-31, wherein the buffer comprises the citrate and/or citric acid in an amount of at least about 100 mM to about 300 mM, optionally wherein the buffer comprises the citrate and/or citric acid in an amount of about 200 mM.

33. The kit of any one of claims 25-32, wherein the citrate and/or citric acid is selected from citric acid, trisodium citrate, potassium citrate, calcium citrate, and/or a hydrate thereof (e.g., tri sodium citrate dihydrate) and/or an anhydrous form thereof, optionally wherein the citrate and/or citric acid is sodium citrate tribasic (e.g., anhydrous sodium citrate tribasic and/or sodium citrate tribasic dihydrate) and citric acid.

34. The kit of any one of claims 25-33, wherein the buffer comprises citric acid in an amount of about 1%, 1.25%, 1.5%, or 1.75% to about 2%, 20.25%, or 2.5% w/w; sodium citrate tribasic and/or tri sodium citrate dihydrate in an amount of about 1.5%, 1.75%, 2%, 20.25%, 2.5%, 20.75%, or 3% to about 30.25%, 3.5%, 30.75%, or 4% w/w; optionally an additional acid (e.g., HCl) and/or base (e.g., NaOH) in an amount sufficient to adjust the pH of the buffer to about 4 to about 6 (e.g., about 4.5); and a remainder of water.

35. The kit of any one of claims 25-34, wherein the buffer consists of water, citric acid, optionally citrate, optionally an additional acid (e.g., HCl), and optionally a base (e.g., NaOH).

36. The kit of any one of claims 24-35, wherein the nitric oxide-releasing active pharmaceutical ingredient has a particle size (e.g., diameter) of about 20 nm to about 30 μm, optionally a mean particle size (e.g., mean diameter) of about 2 μm to about 20 μm.

37. The kit of any one of claims 24-36, wherein the first composition and/or second composition are devoid of a diluent, a co-solvent, a preservative, an antioxidant, a suspending agent, a penetration enhancer, a surfactant, a viscosity-increasing agent, a humectant, a stabilizer, and/or a wetting agent.

38. The kit of any one of claims 24-37, wherein the nitric oxide-releasing active pharmaceutical ingredient comprises a diazeniumdiolate functionalized co-condensed silica network.

39. The kit of any one of claims 24-38, wherein the nitric oxide-releasing active pharmaceutical ingredient comprises a co-condensed silica network comprising diazeniumdiolated methylaminopropyl trimethoxysilane (MAP3-NONOate) and tetraethyl orthosilicate (TEOS).

40. The kit of any one of claims 24-39, wherein the nitric oxide-releasing active pharmaceutical ingredient comprises a co-condensed silica network comprising diazeniumdiolated methylaminopropyl trimethoxysilane (MAP3-NONOate), ethylaminoisobutylsiloxane (EAIB3), and tetraethyl orthosilicate (TEOS).

41. A method of treating and/or preventing an infection (e.g., a viral infection) caused by a pathogen in a subject, the method comprising administering the composition of any one of claims 1-23 to the subject.

42. The method of claim 41, wherein the administering comprises intranasally administering the composition to the subject.

43. The method of claim 41 or 42, further comprising, prior to the administering, combining the nitric oxide-releasing active pharmaceutical ingredient and the buffer to provide the composition, optionally wherein the nitric oxide-releasing active pharmaceutical ingredient and the buffer are present in a device that is configured to combine and/or administer the nitric oxide-releasing active pharmaceutical ingredient and the buffer.

44. The method of any one of claims 41-43, wherein the composition at the time of the administering to the subject has a pH of about 4.5 to about 8.5, optionally wherein the composition has a pH of about 5.5 at the time of the administering to the subject.

45. The method of any one of claims 41-44, wherein the method comprises delivering exogenous nitric oxide to the upper respiratory tract of the subject.

46. The method of any one of claims 41-45, wherein the method reduces or prevents transmission (e.g., viral transmission) of the pathogen to an uninfected subject, optionally compared to the amount of transmission of the pathogen in the absence of a method of the present invention.

47. The method of any one of claims 41-46, wherein the method reduces the amount of the pathogen (e.g., a virus) present in the subject (e.g., in a nasal cavity and/or lung of the subject) by at least about 50%, 60%, 70%, 80%, 90%, or 100% compared to the initial amount of the pathogen present in the subject.

48. The method of any one of claims 41-47, wherein the method reduces the amount of the pathogen (e.g., a virus) present in the lung(s) of the subject and/or reduces or prevents progression of the pathogen into the lung(s) of the subject, optionally compared to the amount of the pathogen and/or progression of the pathogen into the lung(s) of the subject in the absence of a method of the present invention.

49. The method of any one of claims 41-48, wherein the method reduces the severity of the infection in the subject, optionally compared to the severity in the absence of a method of the present invention.

50. The method of any one of claims 41-49, wherein the pathogen is selected from a Coronaviridae virus, Staphylococcus aureus, influenza, or respiratory syncytial virus (RSV).

51. The method of claim 50, wherein the pathogen is severe acute respiratory coronavirus 2 (SARS-CoV-2) or a variant thereof.

52. The method of claim 50, wherein the pathogen is Influenza A/California/7/2009 (H1N1).

53. The method of claim 50, wherein the pathogen is respiratory syncytial virus strain A2 (RSV-A2).

54. The method of any one of claims 41-53, wherein infection is a nosocomial infection.

55. The method of any one of claims 41-54, wherein the method reduces or prevents shedding of the pathogen (e.g., a virus), optionally compared to a method in the absence of the present invention.

56. The method of any one of claims 41-55, wherein the method reduces or inhibits growth and/or replication of the pathogen (e.g., a virus), optionally wherein the method reduces or inhibits growth and/or replication of the pathogen by disrupting a protein function.

57. The method of any one of claims 41-56, wherein the administering comprises delivering gaseous nitric oxide to the lung(s) of the subject.

58. The method of any one of claims 41-57, wherein the method increases oxygenation in the blood of the subject, optionally compared to the oxygen level in the blood prior to administration.

59. The method of any one of claims 41-58, wherein the composition is administered one or more (e.g., 1, 2, 3, 4, 5, 6, 7, or 8) times a day, optionally for 1, 2, 3, 4, 5, 6, 7, 8, 9, 10, 11, 12, 13, 14, or more days.

60. The method of any one of claims 41-59, wherein the composition is administered one, two, or three times a day for about 7 to about 14 days.

61. Use of a composition of any one of claims 1-23 or a kit of any one of claims 24-40 to treat and/or prevent an infection (e.g., a viral infection) in a subject in need thereof.
